(19) United States
(12) Patent Application Publication
(10) Pub. No.: US 2025/0314669 A1
(43) Pub. Date: Oct. 9, 2025

(54) METHODS AND SYSTEMS FOR CHARACTERIZING METAL INTERACTIONS IN ANTIBODY SAMPLES

(71) Applicant: Regeneron Pharmaceuticals, Inc., Tarrytown, NY (US)

(72) Inventors: Xueqing ZHAO, Hastings on Hudson, NY (US); Jikang WU, Chappaqua, NY (US); Haibo QIU, Millwood, NY (US); Ning LI, New Canaan, CT (US)

(73) Assignee: Regeneron Pharmaceuticals, Inc., Tarrytown, NY (US)

(21) Appl. No.: 19/172,979

(22) Filed: Apr. 8, 2025

Related U.S. Application Data
(60) Provisional application No. 63/631,084, filed on Apr. 8, 2024.

(51) Int. Cl. G01N 33/84; G01N 1/40; G01N 33/68

(52) U.S. Cl. CPC G01N 33/84; G01N 1/4077; G01N 33/6848; G01N 33/6854

(57) ABSTRACT

Provided herein are methods for characterizing at least one metal in a sample including a polypeptide or protein of interest. The method may include subject the sample to liquid chromatography separation to form an eluate, subjecting the eluate to inductively coupled plasma mass spectrometry, and subjecting the eluate to high-resolution mass spectrometry. The method may further include identifying the metal and the polypeptide of interest from comparing the results. The polypeptide of interest may be bound to the metal, and/or high molecular weight or low molecular weight species of the polypeptide interest may be bound to the metal.

### CROSS-REFERENCE TO RELATED APPLICATIONS

This application claims the benefit of priority to U.S. Provisional Patent Application No. 63/631,084 filed on Apr. 8, 2024, the entire contents of which are incorporated by referenced herein.

### INTRODUCTION

Trace elements, for example trace metals, are important components in cell culture media for biopharmaceutical production. However, some elements are considered to be impurities in drug products. Sources of elemental impurities include, for example, residuals from elements added intentionally to a cell culture, elements introduced by other cell culture medium ingredients, elements leached from manufacturing equipment, elements present in excipients in a formulation, and elements introduced in containers used in storage and transportation.

Because elemental impurities pose toxicological concerns and do not provide any therapeutic benefit to the patient, their levels in drug products should be controlled within acceptable limits. Elemental impurities can increase the oxidation of a protein product, and can induce the formation of size and charge variants. For example, elemental impurities can induce fragmentation and aggregation of proteins, including therapeutic monoclonal antibodies (mAbs).

Various strategies exist to investigate interactions between mAbs and trace metals or other elements. However, conventional approaches may be time-consuming, may require several mg of protein, may be unable to separate high molecular weight (HMW) and low molecular weight (LMW) species from monomers, and may themselves potentially introduce metal contamination.

Therefore, demand exists for methods and systems for reliable and high throughput analysis of metal-mAb interactions and other elemental-mAb interactions in order to inform the development of safe and effective therapeutics.

### SUMMARY

This disclosure provides methods for characterizing at least one metal in a sample including a polypeptide of interest. In some exemplary aspects, the methods can comprise (a) subjecting a sample including a polypeptide of interest to liquid chromatography (LC) separation to form an eluate; (b) subjecting the eluate to inductively coupled plasma mass spectrometry (ICP-MS) analysis to identify the at least one metal; (c) subjecting the eluate to high-resolution mass spectrometry (HRMS) analysis in parallel to the subjecting of step (b) to identify the polypeptide of interest; and (d) comparing the results of steps (b) and (c) to characterize the at least one metal, wherein a system performing the LC separation is coupled to a system performing the ICP-MS analysis and a system performing the HRMS analysis using a three-way splitter.

Described herein are methods for characterizing at least one metal in a sample including a polypeptide of interest. In some aspects, the method may comprise subjecting a sample including a polypeptide of interest to liquid chromatography (LC) separation to form an eluate, wherein the sample includes at least one metal-bound molecule; subjecting the eluate to inductively coupled plasma mass spectrometry (ICP-MS) analysis to identify at least one metal; subjecting the eluate to high-resolution mass spectrometry (HRMS) analysis to identify the polypeptide of interest; and comparing results of the ICP-MS analysis and the HRMS analysis to characterize the at least one metal. The ICP-MS analysis and the HRMS analysis may be performed in parallel, or sequentially.

Characterizing the at least one metal may comprise determining an isotope of the at least one metal. Characterizing the at least one metal may comprise determining an ionic charge of the at least one metal. Characterizing the at least one metal comprises quantifying an abundance of free metal, small molecule-bound metal, polypeptide-bound metal, or a combination thereof. Characterizing the at least one metal-bound molecule comprises quantifying the at least one metal-bound molecule. Characterizing the at least one metal-bound molecule comprises determining a structure of the at least one metal-bound molecule.

The at least one metal-bound molecule may comprise the polypeptide of interest, at least one high molecular weight (HMW) species of the polypeptide of interest, at least one low molecular weight (LMW) species of the polypeptide of interest, at least one small molecule, or a combination thereof. The at least one metal-bound molecule may comprise a polypeptide, and characterizing the at least one metal-bound molecule comprises determining an amino acid sequence of the polypeptide. The at least one metal-bound molecule may be a truncated protein, and characterizing the at least one metal-bound molecule may comprise determining a clipping site of the at least one metal-bound molecule.

The sample may be a biological sample, such as a serum sample. The at least one metal may be vanadium, iron, cobalt, nickel, copper, zinc, magnesium, aluminum, calcium, titanium, manganese, molybdenum, tungsten, sodium, potassium, cadmium, chromium, silver, or a combination thereof. The polypeptide of interest may be an antibody, a monoclonal antibody, a multispecific antibody, a bispecific antibody, an antibody fragment, a fusion protein, a receptor fusion protein, an antibody-derived protein, an antigen-binding protein, or a variant thereof. The sample may include size variants of the polypeptide of interest. The sample may include a high molecular weight (HMW) species of the polypeptide of interest, a low molecular weight (LMW) species of the polypeptide of interest, or a combination thereof. The polypeptide of interest may be dupilumab.

The method may further comprise, prior to subjecting the eluate to ICP-MS analysis, subjecting the eluate to ultraviolet detection or fluorescence detection. The method may further comprise, prior to subjecting the same to LC separation, subjecting the sample to digestion conditions. Subjecting the sample to digestion conditions may comprise contacting the sample to at least one digestive enzyme. The sample comprises fragments or subunits of said polypeptide of interest.

### BRIEF DESCRIPTION OF THE DRAWINGS

Embodiments of the present disclosure may be implemented in connection with aspects illustrated in the attached drawings. These drawings show different aspects of the present disclosure, and where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. For simplicity and clarity of illustration, the figures depict the general structure and/or manner of construction of the various embodiments/aspects described herein.

FIG. 1 is an illustration depicting an exemplary LC-ICP-MS system, according to aspects of the present disclosure.

FIG. 2A is a chromatogram using sulfur ions to detect mAb-1 signal via LC-ICP-MS, according to aspects of the present disclosure. FIG. 2B is a “zoomed in” view of a portion of FIG. 2A.

FIG. 3A is a chromatogram using copper ions to detect mAb-1 signal via LC-ICP-MS, according to aspects of the present disclosure. FIG. 3B is a “zoomed in” view of a portion of FIG. 3A.

FIG. 4 is a schematic of an exemplary LC-UV-ICP-MS system, according to aspects of the present disclosure.

FIGS. 5A-5F show results of characterizing metal-mAb interactions and metal-small molecule interactions in a mAb-1 sample using exemplary LC-UV-ICP-MS methods, according to aspects of the present disclosure. FIG. 5A is an LC-UV chromatogram at an absorbance of 280 nm. FIG. 5B is a panel of seven LC-ICP-MS chromatograms using chromium (52), manganese (55), iron (56), cobalt (59), nickel (60), copper (63), and zinc (66) ions. FIG. 5C is a base peak chromatogram of full scan at 2500-6000 m/z. FIG. 5D shows an extracted ion chromatogram at 210.0608 m/z (top panel) and an MS1 spectra (bottom panel) for citrate. FIG. 5E shows an extracted ion chromatogram (EIC) at 360.1501 m/z (top panel), an MS1 spectra (second panel), an MS2 spectra at 360.1501 m/z (third panel), and a known reference MS2 spectra (bottom panel) for sucrose. FIG. 5F shows an EIC at 156.0767 m/z (top panel), MS1 spectra (second panel), MS2 spectra at 156.0767 m/z (third panel), and a known reference MS2 spectra (bottom panel) for histidine.

FIG. 6 is a schematic of an exemplary LC-UV-ICP-MS-HRMS system, according to aspects of the present disclosure.

FIG. 7A and FIG. 7B are copper chromatograms generated using exemplary LC-UV-ICP-MS-HRMS methods to characterize metal-mAb interactions in a digested mAb sample, according to aspects of the present disclosure. FIG. 7A shows an overlap of F(ab′)2-Cu and Fc-Cu subdomains when using 0.25 mM ID tubing to connect LC to ICP-MS in a LC-UV-ICP-MS-HRMS system. FIG. 7B shows a separation of F(ab′)2-Cu and Fc-Cu subdomains when using 0.13 mM ID tubing to connect LC to ICP-MS in a LC-UV-ICP-MS-HRMS system.

FIGS. 8A-8H show results of using exemplary LC-UV-ICP-MS-HRMS methods to characterize metal-mAb interactions in a mAb-2 sample, according to aspects of the present disclosure. FIG. 8A is an ICP-MS copper chromatogram in helium mode. FIG. 8B is a base peak mass spectrum in full scan mode with a mass range of 1000-6000 m/z. FIG. 8C is a deconvoluted mass spectrum of the monomer-Cu species. FIG. 8D is a deconvoluted mass spectrum of the low molecular weight-Cu species. FIG. 8E is an ICP-MS copper chromatogram of 1000-6000 m/z. FIG. 8F is an MS1 spectrum of 60-1000 m/z. FIG. 8G is a data-dependent M/SMS (ddMS2) spectrum at 156.0767 m/z. FIG. 8H is a known reference MS2 spectrum for histidine.

FIGS. 9A-9E show results of using exemplary LC-UV-ICP-MS-HRMS methods to characterize metal-mAb interaction in a mAb-3 sample, according to aspects of the present disclosure. FIG. 9A is an ICP-MS iron chromatogram. FIG. 9B is an ICP-MS copper chromatogram. FIG. 9C is a base peak mass spectrum at full scan with 1000-6000 m/z. FIG. 9D is a base peak mass spectrum at full scan with 60-1000 m/z. FIG. 9E is an extracted ion chromatogram at 210.0608 m/z.

FIG. 10 is a panel of five copper chromatograms generated using exemplary LC-UV-ICP-MS-HRMS methods to characterize metal-mAb interactions in five mAb samples (mAb-8, mAb-5, mAb-6, mAb-3, and mAb-2), according to aspects of the present disclosure.

FIGS. 11A-11C show results of using exemplary nSEC-UV-ICP-MS-HRMS methods to characterize metal interactions with a low molecular weight (LMW) species of NISTmAb, according to aspects of the present disclosure. FIG. 11A is an nSEC-UV chromatogram for NISTmAb. FIG. 11B is an nSEC-ICP-MS copper chromatogram. FIG. 11C is an nSEC-ICP-MS sulfur chromatogram.

FIG. 12 is a total ion chromatogram at full scan 2500-6000 m/z of NISTmAb using the exemplary methods described herein, according to aspects of the present disclosure.

FIG. 13A is an averaged raw mass spectrum of NISTmAb using the exemplary methods described herein, according to aspects of the present disclosure. FIG. 13B is a deconvoluted mass spectra of the raw mass spectrum of FIG. 13A.

FIG. 14 is a base peak chromatogram at full scan 60-2000 m/z of NISTmAb using the exemplary methods described herein, according to aspects of the present disclosure.

FIGS. 15A-15C show results of using exemplary methods described herein to characterize metal-molecule interactions, according to aspects of the present disclosure. FIG. 15A is an ICP-MS copper chromatogram of a Cu (II) standard injected onto an SEC column. FIG. 15B is an ICP-MS iron chromatogram of an Fe (III) standard injected onto an SEC column. FIG. 15C is an ICP-MS iron chromatogram of an Fe (III) standard injected onto an SAX column.

FIGS. 16A-16C show results of using exemplary LC-UV-ICP-MS-HRMS methods to characterize copper-histidine interaction in a NISTmAb sample, according to aspects of the present disclosure. FIG. 16A is an MS1 spectrum of the histidine-Cu species. FIG. 16B is an MS2 spectrum of 156.0767 m/z of the histidine-Cu species. FIG. 16C is a known reference MS2 spectrum for histidine.

FIGS. 17A and 17B show results of using exemplary LC-UV-ICP-MS-HRMS methods to characterize copper-protein interactions in IgG4 antibody samples, according to aspects of the present disclosure. FIG. 17A is an ICP-MS copper chromatogram of mAb-1. FIG. 17B is an ICP-MS copper chromatogram of mAb-7.

FIGS. 18A and 18B show results of using exemplary methods described herein to characterize iron-mAb interaction in mAb-2 and mAb-3 samples, according to aspects of the present disclosure. FIG. 18A is a bar graph of total iron concentration for mAb-2 and mAb-3 quantified using direct injection ICP-MS. FIG. 18B is an nSEC-ICP-MS iron chromatogram of mAb-2 and mAb-3.

FIGS. 19A and 19B show results of using exemplary LC-UV-ICP-MS-HRMS methods to characterize iron-mAb interactions in various mAb samples, according to aspects of the present disclosure. FIG. 19A is an ICP-MS iron chromatogram of mAb-3 and mAb-6 samples. FIG. 19B is an ICP-MS iron chromatogram of mAb-3, mAb-5, mAb-6, mAb-7, and mAb-8 samples.

FIGS. 20A and 20B show annotated results of using exemplary LC-UV-ICP-MS-HRMS methods to characterize iron-mAb interactions in various mAb samples, according to aspects of the present disclosure. FIG. 20A shows peak areas of iron bound to mAb species, including monomer-Fe and HMW-Fe species, across the various mAb samples. FIG. 20B shows total iron concentrations across the various mAb samples.

FIGS. 21A-21E show results of using exemplary digestion and LC-UV-ICP-MS-HRMS methods to characterize iron-mAb interactions in a mAb-2 sample, according to aspects of the present disclosure. FIG. 21A is an nSEC-ICP-MS-HRMS iron chromatogram of digested fragments of mAb-2. FIG. 21B is an nSEC-UV chromatogram of digested fragments of mAB-2.

FIG. 21C is a total ion chromatogram at 2500-6000 m/z of digested fragments of mAb-2.

FIG. 21D is a deconvoluted mass spectrum for the F(ab′)₂ fragment peak. FIG. 21E is a deconvoluted mass spectrum for the Fc fragment peak.

FIG. 22 is an ICP-MS iron chromatogram of digested mAb-3 fragments using exemplary digestion and LC-UV-ICP-MS-HRMS method, according to aspects of the present disclosure.

FIGS. 23A and 23B show results of using exemplary LC-UV-ICP-MS-HRMS methods to characterize iron-mAb interactions in mAb-3 and mAb-6 samples with and without EDTA treatment, according to aspects of the present disclosure. FIG. 23A is an iron chromatogram of mAb-3 with and without EDTA treatment. FIG. 23B is an iron chromatogram of mAb-6 with and without EDTA treatment.

FIG. 24 is an iron chromatogram generated using exemplary LC-UV-ICP-MS-HRMS methods to characterize iron-mAb interactions in mAb-3 samples with or without acid treatment, heat treatment, or both, according to aspects of the present disclosure.

FIGS. 25A and 25B show results of using exemplary LC-UV-ICP-MS-HRMS methods to characterize iron-mAb interactions in formulated drug samples (or final drug substances, FDS) of mAb-3 with or without acid treatment, heat treatment, or both, according to aspects of the present disclosure. FIG. 25A shows results before buffer exchange and concentrating, including an SEC-ICP-MS iron chromatogram (top panel) and SEC-UV chromatogram (bottom panel). FIG. 25B shows results after buffer exchange and concentrating, including an SEC-ICP-MS iron chromatogram (top panel) and SEC-UV chromatogram (bottom panel).

FIGS. 26A-26C show results of exemplary LC-UV-ICP-MS-HRMS methods to characterize copper-mAb interactions in various mAb samples with and without EDTA treatment, according to aspects of the present disclosure. FIG. 26A are extracted ion copper chromatograms for mAb-3, mAb-2, mAb-6, mAb-10, and mAb-9. FIG. 26B is an ICP-MS copper chromatogram of mAb-3 with and without EDTA treatment. FIG. 26C is an ICP-MS copper chromatogram of mAb-3 with and without acid treatment, heat treatment, and acid and heat treatments.

FIG. 27 is an ICP-MS iron chromatogram generated using exemplary LC-UV-ICP-MS-HRMS methods to characterize iron-bound species in a mAb-3 sample, according to aspects of the present disclosure.

FIG. 28 is an ICP-MS iron chromatogram generated using exemplary LC-UV-ICP-MS-HRMS methods for characterizing iron-bound small molecule species in a first mAb-3 sample after a first injection or a fourth injection, according to aspects of the present disclosure.

FIGS. 29A-29C are bar graphs of peak area calculations from chromatograms generated using exemplary LC-UV-ICP-MS-HRMS methods to characterize iron-bound small molecule species in two mAb-3 samples across multiple injections, according to aspects of the present disclosure. FIG. 29A shows peak area values for iron-bound small molecule species. FIG. 29B shows peak area values for iron-bound monomer species. FIG. 29C shows peak area values for iron-bound high molecular weight (HMW) species.

FIGS. 30A and 30B show results of using exemplary SAX-ICP-MS-HRMS methods to characterize copper-bound protein species in serum samples, according to aspects of the present disclosure. FIG. 30A is an ICP-MS copper chromatogram of standard human albumin and ceruloplasmin proteins. FIG. 30B is an ICP-MS copper chromatogram of albumin and ceruloplasmin species from a collected human serum sample.

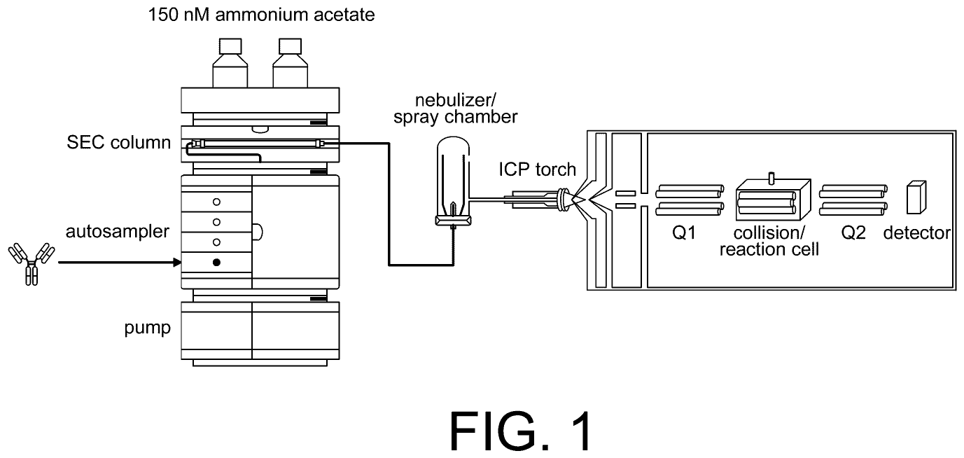

### DETAILED DESCRIPTION

Trace elements, for example trace metals, are important components in cell culture media for biopharmaceutical production. Trace elements facilitate a wide range of intra-and extracellular functions and have a notable effect on cell growth, titers, and product quality. In chemically defined media, some metals, for example sodium (Na), potassium (K), magnesium (Mg), and calcium (Ca), have concentrations in the mM range, while many other metals have sub-μM concentrations. Metal speciation (free salts or chelated) and bioavailability affect cellular uptake of nutrients, metabolism, growth and product quality. Exemplary roles of trace metals in cell culture and biopharmaceutical production are described in Stone et al., “Chemical speciation of trace metals in mammalian cell culture media: looking under the hood to boost cellular performance and product quality”, Curr Opin Biotechnol., 2021, volume 71, pages 216-224, which is incorporated by reference in its entirety. Commonly used chelating agents may include, among others, ethylenediaminetetraacetic acid (EDTA) and citrate.

Some elements may be considered impurities (i.e., elemental impurities) in drug products. Potential sources of elemental impurities include, for example, residuals from elements added intentionally to a cell culture, elements introduced by other cell culture medium ingredients, elements leached from manufacturing equipment such as iron (Fe), chromium (Cr), nickel (Ni), and manganese (Mn) leached from stainless steel bioreactors and magnetic stir bars, elements present in excipients in the formulation such as water for injection, and elements introduced in containers used in storage and transportation, such as tungsten (W) introduced into pre-filled syringes during the creation of the needle hole by a tungsten pin. Because elemental impurities pose toxicological concerns and do not provide any therapeutic benefit to the patient, their levels in drug products should be controlled within acceptable limits. The International Conference on Harmonisation of Technical Requirements for Registration of Pharmaceuticals for Human Use (ICH) Q3D guideline sets forth permitted daily exposures (PDEs) for 24 elemental impurities.

Elemental impurities can increase the oxidation of a protein product, for example a monoclonal antibody (mAb). The presence of iron with a level as low as 20 parts per billion (ppb) may accelerate the oxidation of polysorbate 80 (PS80), a common excipient in pharmaceutical formulations, resulting in the formation of reactive oxygen species (ROS) that can oxidize mAbs. Metal-catalyzed oxidation may induce the formation of acidic variants in mAbs. See, for example, Yang et al., “In-depth characterization of acidic variants induced by metal-catalyzed oxidation in a recombinant monoclonal antibody”, Anal Chem., 2023, volume 95, issue 14, pages 5867-5876, which is hereby incorporated by reference in its entirety.

Exposure to ferrous ions (Fe²⁺) and cupric ions (Cu²⁺) can lead to ion-dependent and site-specific immunoglobulin G1 (IgG1) oxidation, as described in Glover et al., “Physicochemical and biological impact of metal-catalyzed oxidation of IgG1 monoclonal antibodies and antibody-drug conjugates via reactive oxygen species”, mAbs, 2022, volume 14, issue 1, article no. 2122957, pages 1-19, hereby incorporated by reference in its entirety. Elemental impurities can also induce fragmentation and aggregation of proteins, including mAbs. Cu²⁺ is known to mediate an upper hinge region cleavage of IgG1 mostly via a hydrolytic pathway. An Fe³⁺-containing histidine buffer may mediate a visible light-induced heavy chain fragmentation of IgG1 via oxidative cleavage, as described in Zhang et al., “Visible light induces site-specific oxidative heavy chain fragmentation of a monoclonal antibody (IgG1) mediated by an iron (III)-containing histidine buffer”, Mol Pharm., 2023, volume 20, issue 1, pages 650-662, which is hereby incorporated by reference in its entirety. Additionally, zinc ions (Zn²⁺), which may leach from rubber stoppers, can induce IgG1 aggregation via binding to H310 and H435 residues of the fragment crystallizable (Fc) region, as described in Mehta et al., “Metal ion interactions with mAbs: Part 2. Zinc-mediated aggregation of IgG1 monoclonal antibodies”, Pharm Res., 2021, volume 38, pages 1387-1395, which is hereby incorporated by reference in its entirety.

Inductively coupled plasma mass spectrometry (ICP-MS); has been used to investigate interactions between mAbs and trace elements, such as trace metals. Advantages of ICP-MS include increased sensitivity, wide dynamic range, and isotope analysis capabilities, which allows multiple elements to be measured simultaneously in a single analysis. Further, triple quadrupole ICP-MS can operate in MS/MS mode, which offers the potential to improve the removal of spectral interferences compared to single quadrupole ICP-MS. However, while direct injection of a sample in ICP-MS provides absolute quantification of target elements in a sample, it does not show how target elements are distributed among different species or in various forms.

To overcome this challenge, front-end separation techniques are often coupled to ICP-MS. Traditional separation techniques include ultracentrifugal filtration or protein precipitation, followed by acid digestion, and then ICP-MS. Alternatively, liquid chromatography can be coupled to ICP-MS (LC-ICP-MS). Some early examples of LC-ICP-MS include ion pair reversed-phase LC coupled to ICP-MS for element speciation analysis, and size-exclusion chromatography (SEC) coupled to ICP-MS to analyze metal binding protein separated from biological system. See, e.g., Thompson et al., “Inductively coupled plasma mass spectrometric detection for multielement flow injection analysis and elemental speciation by reversed-phase liquid chromatography”, Anal Chem., 1986, volume 58, pages 2541-2548; and Mason et al., “Metalloprotein separation and analysis by directly coupled size exclusion high-performance liquid chromatography inductively coupled plasma mass spectroscopy”, Anal Biochem., 1990, volume 186, pages 187-201, each of which is hereby incorporated by reference in its entirety. More recent studies have described the use of size exclusion chromatography (SEC)-ICP-MS to characterize metal-tagged antibodies and to determine the levels of transition metals in a mAb sample. See, e.g., Mueller et al., “Characterization of metal-tagged antibodies used in ICP-MS-based immunoassays”, Anal Bioanal Chem., 2014, volume 406, issue 1, pages 163-169; and Whitty-Léveillé et al., “Determination of ultra-trace metal-protein interactions in co-formulated monoclonal antibody drug product by SEC-ICP-MS”, mAbs, 2023, volume 15, issue 1, article no. 2199466, pages 1-9, each of which is hereby incorporated by reference in its entirety.

However, the conventional approaches described above are insufficient for various applications. For example, using ultracentrifugal filtration or protein precipitation to separate mAb-binding elements from elements in an excipient is time-consuming, requires several mg of protein, is unable to separate high molecular weight (HMW) and low molecular weight (LMW) species from monomers, and has the potential for introducing metal contamination. Use of a ultracentrifuge filters may release ppb levels of Mg, silicon (Si), Ca, Fe, Ni, and/or Zn. Additionally, conventional LC-ICP-MS only provides time-resolved information at the elemental level, and is incapable of providing structural identification of metal-binding molecules.

To monitor metal-binding molecules at the molecular level, LC-ICP-MS can be simultaneously coupled to an additional detector, such as a mass spectrometer. The simultaneous coupling of RPLC-UV with ICP-MS and time-of-flight (ToF) mass spectrometry was originally reported for the metabolite analysis in urine, where ICP-MS was used to monitor chlorine, sulfur, and bromine, and ToF MS was used to identify the drug metabolites. See, e.g., Corcoran et al., “Directly coupled liquid chromatography with inductively coupled plasma mass spectrometry and orthogonal acceleration time-of-flight mass spectrometry for the identification of drug metabolites in urine: application to diclofenac using chlorine and sulfur detection”, Rapid Comm Mass Spec., 2000, volume 14, issue 24, pages 2377-2384; and Smith et al., “Analysis of a [¹⁴C]-labelled platinum anticancer compound in dosing formulations and urine using a combination of HPLC-ICPMS and flow scintillation counting”, Chromatographia, 2002, volume 55, pages S151-S155, each of which is hereby incorporated by reference in its entirety. However, direct coupling of LC with ICP-MS and an orthogonal mass spectrometry has not been used for protein or native protein analysis. Further, there is no report of coupling LC-ICP-MS with high-resolution mass spectrometry (HRMS).

Therefore, a need exists for sensitive and efficient characterization of trace elements, such as metals, in mAb samples and interactions between elements and components of the sample, including the main mAb species, size variants of the mAb, and small molecules such as excipients. To address this need, a comprehensive approach for characterizing elements and element-bound molecules in a sample was created, including the use of liquid chromatography simultaneously coupled to ICP-MS for elemental analysis and high-resolution mass spectrometry (HRMS) for analysis of proteins and small molecules. While the working examples provided in this disclosure deal with antibodies and antibody fragments, it should be understood that the methods and systems of the present disclosure may be applied to any sample including a trace element and a molecule that may interact with that element, for example a protein, peptide, small molecule, or variant, aggregate, or fragment(s) thereof. Furthermore, while the working examples provided in this disclosure mainly describe metals and metal-bound molecules, it should be understood that the methods and systems of the present disclosure may be applied to any elements of interest in a sample, including non-metals and metalloids, for example, silicon, arsenic, or selenium.

The LC-ICP-MS-HRMS methods and systems of the present disclosure allow for rapid analysis of multiple metals as well as identifying metal-binding molecules simultaneously.

Compared to alternative methods and systems, the methods and systems of the present disclosure do not require complicated sample preparation processes, such as acid digestion. The methods and systems of the present disclosure also consume a small amount of sample (for example, 100-400 μg for each injection), compared to, for example, 3-5 mg per injection for direct injection to ICP-MS.

Unless described otherwise, all technical and scientific terms used herein have the same meaning as commonly understood by one of ordinary skill in the art. Although any methods and materials similar or equivalent to those described herein can be used in the practice or testing, particular methods and materials are now described.

There are many embodiments described and illustrated herein. The present disclosure is neither limited to any single aspect nor embodiment thereof, nor to any combinations and/or permutations of such aspects and/or embodiments. Moreover, each aspect of the present disclosure, and/or embodiments thereof, may be employed alone or in combination with one or more other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate that the embodiment(s) is/are “example” embodiment(s).

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. As used herein, the term polypeptide includes proteins, variants thereof, fragments thereof, and peptides, whether synthetic, naturally occurring, or derived from a larger polypeptide, for example through digestion or truncation. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like.

Proteins of interest or polypeptides of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation”, Biotechnology and Genetic Engineering Reviews, 2012, volume 28, pages 147-176, which is hereby incorporated by reference in its entirety.

In some exemplary aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a Fc/2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen.

An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method. See, e.g., Fan et al., “Bispecific antibodies and their applications”, Journal of Hematology & Oncology, 2015, volume 8, article no. 130, pages 1-14; and Müller et al., “Chapter 11: Bispecific Antibodies”, HANDBOOK OF THERAPEUTIC ANTIBODIES, 2014, pages 265-310 (Eds. Dübel & Reichert, Wiley-VCH Verlag GmbH & Co. KGaA), each of which is hereby incorporated by reference in its entirety. The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the systems and methods disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “sample” can be obtained from any step of a bioprocess, such as cell culture fluid (CCF), harvested cell culture fluid (HCCF), any step in the downstream processing, final concentrated pool (FCP), drug substance (DS), or a drug product (DP) comprising the final formulated product. In some specific aspects, the sample can be selected from any step of the downstream process of clarification, chromatographic production, or filtration. In some specific exemplary aspects, the drug product can be selected from manufactured drug product in the clinic, shipping, storage, or handling.

In some exemplary aspects, the sample is a biological sample. As used herein, the term “biological sample” refers to a sample taken from a living organism, for example a human or non-human mammal. A biological sample may comprise or consist of, for example, whole blood, plasma, serum, saliva, tears, semen, cheek tissue, organ tissue, urine, feces, skin, or hair. A sample may be taken from a patient, for example, a clinical sample. In some exemplary aspects, a sample may be taken from a non-human animal, for example, a preclinical sample. In some exemplary aspects, a sample may be taken from a non-human animal subjected to gene therapy in order to produce at least one protein of interest or polypeptide of interest that may be included in the sample. In some aspects, a sample is a further processed form of any of the aforementioned examples of samples.

As used herein, the term “trace element” refers to a chemical element that is present at a relatively minor quantity or concentration in a sample, as compared to a “bulk element,” which may form structural components of a material, cell or tissue. Trace elements of relevance in biology and the biopharmaceutical industry include, for example, aluminum, arsenic, cadmium, calcium, chromium, cobalt, copper, iodine, iron, magnesium, manganese, mercury, molybdenum, lead, nickel, potassium, tin, titanium, tungsten, selenium, silicon, silver, sodium, vanadium, and zinc.

The following identifies and describes proteins made in cell culture that can be produced and/or characterized according to the present disclosure. Cells comprising the requisite DNA encoding these proteins can be cultured for production according to the present disclosure.

In some exemplary aspects, the protein of interest or polypeptide of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK' (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

For example, for antibody production, some aspects are amenable for research and production use for diagnostics and therapeutics based on all major antibody classes, namely IgG, IgA, IgM, IgD, and IgE. IgG is a preferred class, and includes subclasses IgG1 (including IgG1λ and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g., an anti-PD1 antibody as described in U.S. Patent Application Publication No. 2015/0203579 A1), an anti-Programmed Cell Death Ligand-1 antibody (e.g., an anti-PD-L1 antibody as described in in U.S. Patent Application Publication No. 2015/0203580 A1), an anti-DII4 antibody, an anti-Angiopoietin-2 antibody (e.g., an anti-ANG2 antibody as described in U.S. Pat. No. 9,402,898), an anti-Angiopoietin-Like 3 antibody (e.g., an anti-AngPtl3 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-C5 antibody as described in U.S. Patent Application Publication No. 2015/0313194 A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Patent Application Publication No. 2015/0259423 A1), an anti-Proprotein Convertase Subtilisin Kexin-9 antibody (e.g., an anti-PCSK9 antibody as described in U.S. Pat. No. 8,062,640 or U.S. Patent Application Publication No. 2014/0044730 A1), an anti-Growth And Differentiation Factor-8 antibody (e.g., an anti-GDF8 antibody, also known as anti-myostatin antibody, as described in U.S. Pat. No. 8,871,209 or U.S. Pat. No. 9,260,515), an anti-Glucagon Receptor (e.g., anti-GCGR antibody as described in U.S. Patent Application Publication No. 2015/0337045 A1 or U.S. Patent Application Publication No. 2016/0075778 A1), an anti-VEGF antibody, an anti-ILIR antibody, an interleukin 4 receptor antibody (e.g., an anti-IL4R antibody as described in U.S. Patent Application Publication No. 2014/0271681 A1, U.S. Pat. No. 8,735,095, or U.S. Pat. No. 8,945,559), an anti-interleukin 6 receptor antibody (e.g., an anti-IL6R antibody as described in U.S. Pat. No. 7,582,298, U.S. Pat. No. 8,043,617, or U.S. Pat. No. 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g., anti-IL33 antibody as described in U.S. Patent Application Publication No. 2014/0271658 A1 or U.S. Patent Application Publication No. 2014/0271642 A1), an anti-Cluster of differentiation 3 antibody (e.g., an anti-CD3 antibody, as described in U.S. Patent Application Publication No. 2014/0088295 A1, U.S. Patent Application Publication No. 20150266966 A1, and International Patent Application Publication No. WO 2017/053856 A1), an anti-Cluster of differentiation 20 antibody (e.g., an anti-CD20 antibody as described in U.S. Patent Application Publication No. 2014/0088295 A1, U.S. Patent Application Publication No. 2015/0266966 A1, and U.S. Pat. No. 7,879,984), an anti-CD19 antibody, an anti-CD28 antibody, an anti-Cluster of Differentiation-48 antibody (e.g., anti-CD48 antibody as described in U.S. Pat. No. 9,228,014), an anti-Fel d1 antibody (e.g., as described in U.S. Pat. No. 9,079,948), an anti-influenza virus antibody, an anti-Respiratory syncytial virus antibody (e.g., anti-RSV antibody as described in U.S. Patent Application Publication No. 2014/0271653 A1), an anti-Middle East Respiratory Syndrome virus antibody (e.g., an anti-MERS-COV antibody as described in U.S. Patent Application Publication No. 2015/0337029 A1), an anti-Ebola virus antibody (e.g., as described in U.S. Patent Application Publication No. 2016/0215040 A1), an anti-Zika virus antibody, an anti-Severe Acute Respiratory Syndrome (SARS) antibody (e.g., an anti-SARS-COV antibody), an anti-COVID-19 antibody (e.g., an anti-SARS-COV-2 antibody), an anti-Lymphocyte Activation Gene 3 antibody (e.g., an anti-LAG3 antibody, or an anti-CD223 antibody), an anti-Nerve Growth Factor antibody (e.g., an anti-NGF antibody as described in U.S. Patent Application Publication No. 2016/0017029 A1, U.S. Pat. No. 8,309,088, and U.S. Pat. No. 9,353,176), and an anti-Activin A antibody. In some aspects, the bispecific antibody is selected from the group consisting of an anti-CD3×anti-CD20 bispecific antibody (as described in U.S. Patent Application Publication No. 2014/0088295 A1 and U.S. Patent Application Publication No. 2015/0266966 A1), an anti-CD3×anti-Mucin16 bispecific antibody (e.g., an anti-CD3×anti-Muc16 bispecific antibody), an anti-CD3×BCMA bispecific antibody, and an anti-CD3×anti-Prostate-specific membrane antigen bispecific antibody (e.g., an anti-CD3×anti-PSMA bispecific antibody). Also included are a MetxMet antibody, an agonist antibody to NPR1, an LEPR agonist antibody, a MUC16×CD28 antibody, a GITR antibody, an IL-2Rg antibody, an EGFR×CD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies may be cultured. In some aspects, the protein of interest or polypeptide of interest is selected from the group consisting of Alirocumab, Atoltivimab, Maftivimab, Odesivimab, Odesivimab-ebgn, Casirivimab, Imdevimab, Cemiplimab and Cemiplimab-rwlc (human IgG4 monoclonal antibody that binds to PD-1), Sarilumab, Fasinumab, Nesvacumab, Dupilumab (human monoclonal antibody of the IgG4 subclass that binds to the IL-4R alpha (a) subunit and thereby inhibits Interleukin 4 (IL-4) and Interleukin 13 (IL-13) signaling), Trevogrumab, Evinacumab, Evinacumab-dgnb, Fianlimab, Garetosmab, Itepekimab, Odrononextamab, Pozelimab, Rinucumab, and modifications, truncations, and variations thereof.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the systems and methods of the present disclosure also are applicable to the production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

In the United States, a biosimilar is currently described as (A) a biological product is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be facilitated with the methods and systems of the present disclosure.

In some aspects, the protein of interest or polypeptide of interest is a recombinant protein that contains an Fc moiety and another domain, (e.g., an Fc-fusion protein). In some aspects, an Fc-fusion protein is a receptor Fc-fusion protein, which contains one or more extracellular domain(s) of a receptor coupled to an Fc moiety. In some aspects, the Fc moiety comprises a hinge region followed by a CH2 and CH3 domain of an IgG. In some aspects, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-1RAcP ligand binding region fused to the II-1R1 extracellular region fused to Fc of hIgG1; see U.S. Pat. No. 6,927,004, which is herein incorporated by reference in its entirety), or a VEGF trap (e.g., aflibercept or ziv-aflibercept, which contains the Ig domain 2 of the VEGF receptor Flt1 fused to the Ig domain 3 of the VEGF receptor Flk1 fused to Fc of hIgG1; see U.S. Pat. No. 7,087,411 and U.S. Pat. No. 7,279,159). In other aspects, an Fc-fusion protein is a ScFv-Fc-fusion protein, which contains one or more of one or more antigen-binding domain(s), such as a variable heavy chain fragment and a variable light chain fragment, of an antibody coupled to an Fc moiety. In at least one aspect, the protein of interest comprises a combination of any of the foregoing.

In some aspects, a sample can be prepared prior to or following enrichment steps, separation steps, and/or analysis steps. Preparation steps can include alkylation, reduction, denaturation, digestion, derivatization, and/or deglycosylation.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some aspects, non-reduced preparation may be used, for example, in order to preserve an endogenous disulfide bond between Fab arms of an antibody or antibody-derived protein. In other aspects, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein or polypeptide. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein or polypeptide. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C), outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switzar et al., “Protein Digestion: An Overview of the Available Techniques and Recent Developments”, J Proteome Res., 2013, volume 12, issue 3, pages 1067-1077, which is hereby incorporated by reference in its entirety.

In some exemplary aspects, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FABRICATOR® or FABRICATOR Z®.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC). In some aspects, a sample can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, ultraviolet (UV) detection or fluorescence detection.

In some exemplary aspects, the methods and systems of the present disclosure include the use of size exclusion chromatography. Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material may include a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

In some exemplary aspects, the mobile phase used to obtain an eluate from size exclusion chromatography can comprise a volatile salt. In some specific aspects, the mobile phase can comprise ammonium acetate, ammonium bicarbonate, or ammonium formate, or combinations thereof.

Online coupling of SEC with direct MS detection under near native conditions (native SEC-MS) has gained interest in recent years to study mAb HMW species. See, e.g., Rouby et al., supra; Ehkirch et al., “Hyphenation of size exclusion chromatography to native ion mobility mass spectrometry for the analytical characterization of therapeutic antibodies and related products”, Journal of Chromatography B, 2018, volume 1086, pages 176-183; and Haberger et al., “Rapid characterization of biotherapeutic proteins by size-exclusion chromatography coupled to native mass spectrometry”, mAbs, 2016, volume 8, issue 2, pages 331-339, each of which is hereby incorporated by reference in its entirety. Using MS-compatible mobile phases that can preserve protein conformation and non-covalent interactions, native SEC-MS (nSEC-MS) can provide rapid and improved identification of size variants based on accurate mass measurement.

In some exemplary aspects, the methods and systems of the present disclosure include the use of anion exchange chromatography, including strong anion exchange chromatography. Anion exchange chromatography is based on ionic interactions between the binding entity (protein of interest, variant, or impurity) and the functional group immobilized on the chromatographic media. Performance may be a function of the mobile phase, the functional group, and the resin backbone.

As used herein, the term “inductively coupled plasma mass spectrometry” (ICP-MS) refers to a technique including the use of inductively coupled plasma to ionize a sample, which is then detected by MS. ICP-MS is capable of creating atomic and small polyatomic ions, which allows for the detection of elements such as metals at very low concentrations, as well as differentiation between different isotopes of the same element. Liquid samples are first nebulized in a sample introduction system, creating a fine aerosol that is then transferred to argon plasma. The high-temperature plasma atomizes and ionizes the sample, generating ions which are extracted through the interface region and into a set of electrostatic lenses called the ion optics. The ion optics focus and guide the ion beam into the mass analyzer, which separates ions according to their mass-to-charge ratio (m/z), and these ions are measured at a detector. Elements that can be analyzed using ICP-MS include any element except hydrogen, helium, fluorine, neon, or argon. Particular elements of interest for biopharmaceutical purposes include, for example, aluminum, arsenic, cadmium, calcium, chromium, cobalt, copper, iron, magnesium, manganese, molybdenum, nickel, potassium, titanium, tungsten, selenium, silicon, silver, sodium, vanadium, and zinc.

In some exemplary aspects, the ICP-MS of the present disclosure is triple quadrupole ICP-MS. The mass analyzer includes a first quadrupole mass filter (Q1), a collision/reaction cell, and a second quadrupole mass filter (Q2). The ions are passed into the Q1, which can be configured to allow all ions to pass or only ions of a particular mass-to-charge ratio (m/z). Ions then enter the collision/reaction cell, where special interferences can be resolved using reactive cell gasses or helium collision mode. Ions are then passed into the Q2, which separates ions according to their m/z and passes only the target analyte ions/product to the detector.

As used herein, the term “mass spectrometer” includes a device capable of identifying specific molecular species and measuring their accurate masses. The term is meant to include any molecular detector with which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary aspects, the methods and systems of the present disclosure may include high-resolution mass spectrometry (HRMS), which allows for detection of analytes with a high resolution and high degree of mass accuracy by taking advantage of sensitive mass spectrometry instruments. The high resolution in HRMS may be achieved by using advanced mass analyzers, such as time-of-flight (TOF), Orbitrap, and Fourier Transform Ion Cyclotron Resonance (FT-ICR). These analyzers can measure the mass-to-charge ratio of ions with a very high degree of accuracy, often to four decimal places or more. This allows for the identification of compounds based on their exact mass, providing a high level of specificity in the analysis.

In some aspects, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the methods and systems of the present disclosure include the use of a tandem mass spectrometer equipped with a quadrupole mass filter, a collision cell, and an Orbitrap mass analyzer, for example the Thermo Exploris™ 240. MS² may be used to aid in the structural identification of small molecules that bind to metals or other elements.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary aspects, the mass spectrometer may use a microflow nanospray ion source, for example NEWOMICS® MnESI (microflow nanospray electrospray ionization) ion source, with M3 emitter. The ion source has multiple nozzles working together to split a single microflow stream evenly into multiple nanoflows. The MnESI source can be linked to a high-flow LC system using a polyether ether ketone (PEEK) T-splitter. This configuration allows a microflow to enter the MnESI, while the remaining analytical flow is directed towards the ICP-MS. The use of a PEEK splitter, as opposed to a metal splitter, prevents potential metal contamination. PEEK is a high-performance thermoplastic that is resistant to chemical interactions, ensuring that it doesn't introduce undesirable metal ions into the system that may interfere with analysis.

In some aspects, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range. See, e.g., Picotti et al., “Selected reaction monitoring-based proteomics: workflows, potential, pitfalls and future directions”, Nature Methods, 2012, volume 9, pages 555-566, which is hereby incorporated by reference in its entirety. MRM can be typically performed with triple quadrupole mass spectrometers wherein a precursor ion corresponding to the selected small molecules/peptides is selected in the first quadrupole and a fragment ion of the precursor ion is selected for monitoring in the third quadrupole. See, e.g., Choi et al., “Targeted human cerebrospinal fluid proteomics for the validation of multiple Alzheimer's disease biomarker candidates”, Journal of Chromatography B, 2013, volume 930, pages 129-135, which is hereby incorporated by reference in its entirety.

In some aspects, LC-MS can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution.

Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In some aspects, native MS allows for better spatial resolution compared to non-native MS, improving detection of biotransformation products of a therapeutic protein. For detailed review on native MS, see Erba et al., “The emerging role of native mass spectrometry in characterizing the structure and dynamics of macromolecular complexes”, Protein Science, 2015, volume 24, pages 1176-1192, which is hereby incorporated by reference in its entirety.

This disclosure provides methods for characterizing at least one element in a sample including a polypeptide of interest. In some exemplary aspects, the methods can comprise (a) subjecting a sample including a polypeptide of interest to liquid chromatography (LC) separation to form an eluate; (b) subjecting the eluate to inductively coupled plasma mass spectrometry (ICP-MS) analysis to identify the at least one element; (c) subjecting the eluate to high-resolution mass spectrometry (HRMS) analysis in parallel to the subjecting of step (b) to identify the polypeptide of interest; and (d) comparing the results of steps (b) and (c) to characterize the at least one element, wherein a system performing the LC separation is coupled to a system performing the ICP-MS analysis and a system performing the HRMS analysis using a three-way splitter.

This disclosure also provides methods for characterizing at least one element-bound molecule in a sample including a polypeptide of interest. In some exemplary aspects, the methods can comprise (a) subjecting a sample including a polypeptide of interest to liquid chromatography (LC) separation to form an eluate, wherein the sample includes at least one element-bound molecule; (b) subjecting the eluate to inductively coupled plasma mass spectrometry (ICP-MS) analysis to identify at least one element; (c) subjecting the eluate to high-resolution mass spectrometry (HRMS) analysis in parallel to the subjecting of step (b) to identify at least one molecule having substantially the same retention time as the at least one element; and (d) characterizing the at least one element-bound molecule using the results of step (b) and step (c), wherein a system performing the LC separation is coupled to a system performing the ICP-MS analysis and a system performing the HRMS analysis using a three-way splitter.

In one aspect, characterizing the at least one element comprises determining or identifying the at least one element. In another aspect, characterizing the at least one element comprises determining an ionic charge of the at least one element. In a further aspect, characterizing the at least one element comprises determining an isotope of the at least one element. In an additional aspect, characterizing the at least one element comprises quantifying the at least one element, optionally wherein characterizing the at least one element comprises quantifying the at least one element for at least two retention times.

In one aspect, characterizing the at least one element comprises identifying and/or quantifying at least one molecule bound to the at least one element, optionally wherein the at least one molecule comprises the polypeptide of interest, at least one HMW species of the polypeptide of interest, at least one LMW species of the polypeptide of interest, and/or at least one small molecule. In another aspect, characterizing the at least one element comprises quantifying an abundance of free element, small molecule-bound element, and/or polypeptide-bound element.

In one aspect, the at least one element-bound molecule comprises the polypeptide of interest, at least one HMW species of the polypeptide of interest, at least one LMW species of the polypeptide of interest, and/or at least one small molecule. In another aspect, the at least one element-bound molecule is a polypeptide and characterizing the at least one element-bound molecule comprises determining an amino acid sequence of the at least one element-bound molecule.

In one aspect, characterizing the at least one element-bound molecule comprises quantifying the at least one element-bound molecule. In another aspect, characterizing the at least one element-bound molecule comprises determining a structure of the at least one element-bound molecule. In a further aspect, the at least one element-bound molecule is a truncated protein and characterizing the at least one element-bound molecule comprises determining a clipping site of the at least one element-bound molecule.

In one aspect, the method further comprises subjecting the eluate to ultraviolet detection or fluorescence detection prior to subjecting the eluate to ICP-MS analysis. In a specific aspect, the method further comprises characterizing the at least one element by comparing the results of the ultraviolet detection or fluorescence detection to the results of the ICP-MS analysis and/or the HRMS analysis. In another specific aspect, the method further comprises characterizing the at least one element-bound molecule by comparing the results of the ultraviolet detection or fluorescence detection to the results of the ICP-MS analysis and/or the HRMS analysis.

In one aspect, the method further comprises comparing the characterization of the at least one element in a first sample to a characterization of at least one element in a second sample. In another aspect, the method further comprises comparing the characterization of the at least one element-bound molecule in a first sample to a characterization of at least one element-bound molecule in a second sample.

In one aspect, the first sample has not been subjected to treatment to dissociate element-polypeptide interactions and the second sample has been subjected to treatment to dissociate element-polypeptide interactions. In a specific aspect, the treatment comprises incubating the second sample with a chelator, subjecting the second sample to acidic conditions, and/or subjecting the second sample to heating conditions. In a more specific aspect, incubating the second sample with a chelator comprises incubating the second sample with ethylenediaminetetraacetic acid, optionally wherein the incubating is performed for about one hour. In another specific aspect, subjecting the second sample to acidic conditions comprises incubating the second sample with acetic acid, optionally wherein the acetic acid is at a concentration of about 5 mM. In a further specific aspect, subjecting the second sample to heating conditions comprises subjecting the second sample to temperatures from 50 to 100° C., from 70 to 90° C., about 70° C., about 75° C., about 80° C., about 85° C., or about 90° C. for from 1 minute to 30 minutes, from 5 minutes to 20 minutes, about 5 minutes, about 10 minutes, about 15 minutes, or about 20 minutes. In another specific aspect, the first sample has been subjected to fewer injections in the system performing the LC separation compared to the second sample. In a further specific aspect, the first sample is from a first step in a production process for the polypeptide of interest and the second sample is from a second step in the production process for the polypeptide of interest. In an additional specific aspect, the first sample includes a first polypeptide of interest and the second sample includes a second polypeptide of interest.

In one aspect, the polypeptide of interest is a therapeutic polypeptide and the sample is a sample from a production or purification process, a chromatography pool, a final concentrated pool, a drug substance, or a drug product. In another aspect, the sample is a biological sample, optionally wherein the biological sample is a serum sample.

In one aspect, the at least one element is a contaminant, an element component of a cell culture medium, an element contaminant from manufacturing equipment, an element component of a formulation, and/or an element contaminant from a storage or transportation container. In another aspect, the at least one element is a metal, a metalloid, or a non-metal. In a specific aspect, the at least one metal is selected from a group consisting of vanadium, iron, cobalt, nickel, copper, zinc, magnesium, aluminum, calcium, titanium, manganese, molybdenum, tungsten, sodium, potassium, cadmium, chromium, silver, and combinations thereof. In another specific aspect, the at least one element is selected from a group consisting of aluminum, arsenic, cadmium, calcium, chromium, cobalt, copper, iron, magnesium, manganese, molybdenum, nickel, potassium, titanium, tungsten, selenium, silicon, silver, sodium, vanadium, zinc, and combinations thereof.

In one aspect, the polypeptide of interest is an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, or a variant thereof.

In one aspect, the liquid chromatography separation comprises reversed phase liquid chromatography, ion exchange chromatography, anion exchange chromatography, strong anion exchange chromatography, cation exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof. In another aspect, the liquid chromatography separation is performed under native conditions.

In one aspect, a nebulizer for the ICP-MS analysis is a quartz concentric nebulizer. In another aspect, an internal diameter (I.D.) of tubing connecting the three-way splitter to the system performing the ICP-MS analysis is from 0.1 mM to 0.2 mM, or about 0.13 mM.

In one aspect, the ICP-MS analysis is performed in oxygen mode or helium mode.

In one aspect, the system performing the HRMS analysis comprises an electrospray ionization mass spectrometer, nano-electrospray ionization mass spectrometer, or a triple quadrupole mass spectrometer. In another aspect, the system performing the HRMS analysis comprises a microflow-nanospray electrospray ionization source.

In one aspect, the HRMS analysis is performed with a mass range of about 60-1000 m/z and/or about 1000-6000 m/z.

In one aspect, the sample comprises size variants of the polypeptide of interest. In another aspect, the sample comprises high molecular weight (HMW) species and/or low molecular weight (LMW) species of the polypeptide of interest.

In one aspect, the sample comprises at least one small molecule, optionally wherein the at least one small molecule is histidine, citrate, or sucrose.

In one aspect, the three-way splitter directs a majority of eluate to the system performing ICP-MS analysis, optionally wherein a ratio of eluate directed to the system performing HRMS analysis to eluate directed to the system performing ICP-MS analysis is from 1:5 to 1:50, from 1:10 to 1:30, about 1:10, about 1:15, about 1:20, about 1:25, or about 1:30.

In one aspect, the method further comprises subjecting the sample to digestion conditions prior to the LC separation. In a specific aspect, subjecting the sample to digestion conditions comprises contacting the sample to at least one digestive enzyme. In a more specific aspect, the at least one digestive enzyme is selected from a group consisting of pepsin, trypsin, Tryp-N, chymotrypsin, Lys-N, Lys-C, Asp-N, Arg-C, Glu-C, papain, IdeS, or a variant thereof.

In one aspect, the sample comprises fragments or subunits of the polypeptide of interest. In a specific aspect, the polypeptide of interest is an antibody or antibody-derived protein and the fragments or subunits comprise Fab fragments, Fab′ fragments, Fab₂ fragments, F(ab′)₂ fragments, Fc fragments, Fc/2 fragments, Fv fragments, Fd fragments, and/or Fd′ fragments.

This disclosure further provides systems for characterizing at least one element in a sample including a polypeptide of interest. In some exemplary aspects, the systems can comprise a liquid chromatography system capable of receiving a sample including a polypeptide of interest coupled to a first arm of a three-way splitter, wherein a second arm of the three-way splitter is coupled to an ICP-MS system capable of identifying the at least one element and a third arm of the three-way splitter is coupled to a HRMS system capable of identifying the polypeptide of interest.

This disclosure additionally provides systems for characterizing at least one element-bound molecule in a sample including a polypeptide of interest. In some exemplary aspects, the systems can comprise a liquid chromatography system capable of receiving a sample including a polypeptide of interest coupled to a first arm of a three-way splitter, wherein a second arm of the three-way splitter is coupled to an ICP-MS system capable of identifying at least one element and a third arm of the three-way splitter is coupled to a HRMS system capable of identifying at least one molecule having substantially the same retention time as the at least one element.

In one aspect, characterizing the at least one element comprises determining or identifying the at least one element. In another aspect, characterizing the at least one element comprises determining an ionic charge of the at least one element. In a further aspect, characterizing the at least one element comprises determining an isotope of the at least one element. In an additional aspect, characterizing the at least one element comprises quantifying the at least one element, optionally wherein characterizing the at least one element comprises quantifying the at least one element for at least two retention times.

In one aspect, characterizing the at least one element comprises identifying and/or quantifying at least one molecule bound to the at least one element, optionally wherein the at least one molecule comprises the polypeptide of interest, at least one HMW species of the polypeptide of interest, at least one LMW species of the polypeptide of interest, and/or at least one small molecule. In another aspect, characterizing the at least one element comprises quantifying an abundance of free element, small molecule-bound element, and/or protein-bound element.

In one aspect, the at least one element-bound molecule comprises the polypeptide of interest, at least one HMW species of the polypeptide of interest, at least one LMW species of the polypeptide of interest, and/or at least one small molecule. In another aspect, the at least one element-bound molecule is a protein and characterizing the at least one element-bound molecule comprises determining an amino acid sequence of the at least one element-bound molecule.

In one aspect, characterizing the at least one element-bound molecule comprises quantifying the at least one element-bound molecule. In another aspect, characterizing the at least one element-bound molecule comprises determining a structure of the at least one element-bound molecule. In a further aspect, the at least one element-bound molecule is a truncated protein and characterizing the at least one element-bound molecule comprises determining a clipping site of the at least one element-bound molecule.

In one aspect, the system further comprises an ultraviolet detector, fluorescence detector, or diode array detector coupled to the liquid chromatography system.

In one aspect, the at least one element is a contaminant, an element component of a cell culture medium, an element contaminant from manufacturing equipment, an element component of a formulation, and/or an element contaminant from a storage or transportation container. In another aspect, the at least one element is a metal, a metalloid, or a non-metal. In a specific aspect, the at least one metal is selected from a group consisting of vanadium, iron, cobalt, nickel, copper, zinc, magnesium, aluminum, calcium, titanium, manganese, molybdenum, tungsten, sodium, potassium, cadmium, chromium, silver, and combinations thereof. In another specific aspect, the at least one element is selected from a group consisting of aluminum, arsenic, cadmium, calcium, chromium, cobalt, copper, iron, magnesium, manganese, molybdenum, nickel, potassium, titanium, tungsten, selenium, silicon, silver, sodium, vanadium, zinc, and combinations thereof.

In one aspect, the polypeptide of interest is an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, or a variant thereof.

In one aspect, the liquid chromatography system comprises reversed phase liquid chromatography, ion exchange chromatography, anion exchange chromatography, strong anion exchange chromatography, cation exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof. In another aspect, the liquid chromatography system is configured to operate under native conditions.

In one aspect, a nebulizer for the ICP-MS system is a quartz concentric nebulizer. In another aspect, an internal diameter (I.D.) of tubing connecting the three-way splitter to the ICP-MS system is from 0.1 mM to 0.2 mM, or about 0.13 mM.

In one aspect, the ICP-MS system is configured to function in oxygen mode or helium mode.

In one aspect, the HRMS system comprises an electrospray ionization mass spectrometer, nano-electrospray ionization mass spectrometer, or a triple quadrupole mass spectrometer. In another aspect, the HRMS system comprises a microflow-nanospray electrospray ionization source.

In one aspect, the sample comprises size variants of the polypeptide of interest. In another aspect, the sample comprises high molecular weight (HMW) species and/or low molecular weight (LMW) species of the polypeptide of interest. In a further aspect, the sample comprises at least one small molecule, optionally wherein the at least one small molecule is histidine, citrate, or sucrose.

In one aspect, the three-way splitter is configured to direct a majority of eluate to the ICP-MS system, optionally wherein a ratio of eluate directed to the HRMS system to eluate directed to the ICP-MS system is from 1:5 to 1:50, from 1:10 to 1:30, about 1:10, about 1:15, about 1:20, about 1:25, or about 1:30.

In one aspect, the sample comprises fragments or subunits of the polypeptide of interest. In a specific aspect, the polypeptide of interest is an antibody or antibody-derived protein and the fragments or subunits comprise Fab fragments, Fab′ fragments, Fab₂ fragments, F(ab′)₂ fragments, Fc fragments, Fc/2 fragments, Fv fragments, Fd fragments, and/or Fd′ fragments.

It is understood that the present disclosure is not limited to any of the aforethe protein(s), protein(s) of interest, polypeptide(s) of interest, recombinant protein(s), antibody(s), antibody fragment(s), small molecule(s), cell(s), cell type(s), cell line(s), cell culture media, protein alkylating agent(s), protein denaturing agent(s), protein reducing agent(s), digestive enzyme(s), sample(s), chromatographic method(s), ICP-MS method(s), metal(s), element(s), HRMS method(s), nebulizer(s), mass spectrometer(s), temperature(s), or concentration(s), and any protein(s), protein(s) of interest, polypeptide(s) of interest, recombinant protein(s), antibody(s), antibody fragment(s), small molecule(s), cell(s), cell type(s), cell line(s), cell culture media, protein alkylating agent(s), protein denaturing agent(s), protein reducing agent(s), digestive enzyme(s), sample(s), chromatographic method(s), ICP-MS method(s), metal(s), element(s), HRMS method(s), nebulizer(s), mass spectrometer(s), temperature(s), or concentration(s) can be selected by any suitable means.

The present disclosure will be more fully understood by reference to the following examples. The following examples include exemplary methods and systems. Those of ordinary skill in the art would understand that other methods and systems, not described in the examples, are within the scope of the present disclosure.

### EXAMPLES

Materials. The following monoclonal antibodies were produced by Regeneron (Tarrytown, NY): mAb-1 (IgG4 bispecific antibody), mAb-2 (IgG1 antibody), mAb-3 (IgG1 antibody), mAb-5 (IgG1 antibody), mAb-6 (IgG1 antibody), mAb-7 (IgG4 bispecific antibody), mAb-8 (IgG1 antibody), mAb-9 (IgG4 antibody), and mAb-10 (IgG4 antibody). NISTmAb, a humanized IgG1κ monoclonal antibody, was purchased from the National Institute of Standards and Technology (Gaithersburg, MD) and used as a standard. FABRICATOR® enzyme, an IgG hinge digestive enzyme, was purchased from Genovis (Cambridge, MA).

Sample Preparation. The monoclonal antibody samples for metal-mAb interaction analysis were diluted in 150 mM ammonium acetate buffer with a final concentration of 10 mg/mL. To prepare the IdeS digested mAb samples, an aliquot of mAb-2 and mAb-3 DS samples was each subjected to digestion with FABRICATOR® (1.25 units per 1 μg protein) in 150 mM ammonium acetate at 37° C. for 1 hour, to generate the F(ab′)₂ and Fc fragments.

LC-UV-ICPMS-HRMS Methods. Native SEC (nSEC) chromatography was performed on a 1290 Infinity II UHPLC system (AGILENT®, Santa Monica, CA) equipped with a diode array detector (DAD). The mAb samples were eluted and separated on an ACQUITY® UPLC Protein BEH SEC Column (200 Å, 1.7 μm, 4.6×300 mm, Waters, Millford, MA) with an isocratic flow of 150 mM ammonium acetate at 0.2 mL/min. The column compartment temperature was set to 30° C. To enable online ICP-MS analysis and HRMS analysis simultaneously, LC flow was divided by a PEEK T-splitter into a microflow (<10 μL/mL) for nanoelectrospray ionization (NSI)-MS detection and a remaining high flow for metal analysis. An 8900 ICP-QQQ (AGILENT®, Santa Monica, CA) equipped with a quartz concentric nebulizer was used for metal analysis. High resolution MS analysis was carried out on an Orbitrap EXPLORIS™ 240 mass spectrometer (THERMO FISHER SCIENTIFIC®, San Jose, CA) equipped with a microflow-nanospray electrospray ionization (MnESI) source and a microfabricated monolithic multinozzle (M3) emitter (NEWOMICS®, Berkley, CA).

Total Iron Quantification by ICP-MS. Total iron analysis of each mAb sample involved a hot plate-assisted digestion process, as previously described by Astolfi et al., “Optimization and validation of a fast digestion method for the determination of major and trace elements in breast milk by ICP-MS”, Analytica Chimica Acta, 2018, volume 1040, pages 49-62, which is hereby incorporated by reference in its entirety. Polypropylene digestion tubes were subjected to a 24-hour immersion in a 5% (v/v) nitric acid (HNO₃) solution to mitigate potential metal contamination. Following this, the tubes were rinsed with Milli-Q water and dried in a laminar flow hood. Approximately 50 mg of mAb samples were then introduced to the digestion tubes containing a nitric acid to hydrogen peroxide (HNO₃:H₂O₂) ratio of 2:1. The tubes were loosely capped and heated at 70° C. for 2 hours in an EPPENDORF THERMOMIXER® heat block. Post-digestion, the resultant clear acid solutions were diluted with ultrapure water to achieve a final concentration of 10.0% (v/v) HNO₃. The digested sample solutions were appropriately diluted as needed and subjected to total iron analysis using an 8900 ICP-QQQ (AGILENT®, Santa Clara, CA) equipped with an octopole reaction system (ORS) collision/reaction cell technology to minimize spectral interferences. The continuous sample introduction system comprised an AGILENT® integrated autosampler (I-AS), a quartz torch with a 2.5-mm diameter injector, a Scott double-pass spray chamber, and nickel cones. The parameters for total iron content determination included a plasma Rf power of 1,550 W, a nebulizer operating at an argon flow rate of 1.07 L/min, a peristaltic pump speed of 0.3 rps, and a sample volume of 2 mL. Iron quantification was performed under H₂ reaction gas mode (flow rate of 6.0 mL/min) at m/z of ⁵⁶Fe (56→56) with an integration time of 0.3 s. An internal standard of ⁴⁵Sc was utilized to monitor potential variations due to instrument drift and/or matrix effects. External calibration standards were meticulously prepared by diluting the Agilent single-element iron standard stock solution (1 g/L) with 7.0% (v/v) HNO₃ acid, yielding solutions of 0.05 μg/L, 0.1 μg/L, 0.5 μg/L, 1 μg/L, 5 μg/L, 10 μg/L, 50 μg/L, and 200 μg/L. The total iron content was subsequently computed and expressed in terms of ng/g of protein.

Data Analysis. ICP-MS chromatograms and ICP-MS data for total iron quantification were processed and exported using AGILENT®MassHunter Software. UV chromatograms were processed and exported using AGILENT® OpenLAb software. Intact mass spectra of nSEC-MS analysis were deconvoluted using Intact Mass software from Protein Metrics (Cupertino, CA). The acquired data at small molecule m/z range was processed using Compound Discoverer software, and the mzCloud, mzVault with an in-house MS/MS database, and ChemSpider databases were used for annotation.

### Example 1: Development of LC-ICP-MS Methods for Metal-mAb Analysis

In order to address the needs for fast, sensitive and efficient determination of interactions between proteins of interest and trace elements (e.g., metal-mAb interactions), a LC-ICP-MS method was developed using an AGILENT® 1290 II Infinity LC system and an AGILENT® ICP-MS 8900 system. A native size exclusion chromatography (nSEC) system was coupled with ICP-MS to determine Cu-mAb interactions under native conditions.

The recombinant bispecific IgG1 antibody mAb-1 was compared to the antibody standard, NISTmAb. To confirm the identity of protein signals in copper chromatograms, sulfur(S) chromatograms were generated for the samples and used to monitor protein signals. ICP-MS, when operated in the O₂ reaction mode, can be used to detect sulfur by removing the spectral interferences (i.e., main interfering ions) of ³²S that are formed inside the plasma, (mainly ¹⁶O¹⁶O⁺). Main interfering ions for each of sulfur isotopes are shown in Table 1, below.

TABLE 1

| Isotope | Abundance | Main Interfering Ions |
|---|---|---|
| ³²S | 94.93 | ¹⁶O¹⁶O⁺; ¹⁴N¹⁸O⁺; ¹⁵N¹⁶O¹H⁺; ¹⁴N¹⁶O¹H₂⁺ |
| ³³S | 0.76 | ¹⁶O¹⁶O¹H⁺; ¹⁴N¹⁸O1H⁺; ¹⁵N¹⁸O⁺ |
| ³⁴S | 4.29 | ¹⁶O¹⁸O⁺; ¹⁶O¹⁷O¹H⁺; ¹⁶O¹⁶O¹H₂⁺; ¹⁵N¹⁸O¹H⁺ |
| ³⁶S | 0.02 | ³⁶Ar |

Sulfur was determined as the oxide ion ³²S¹⁶O⁺ at 32→48 and as the oxide ion ³⁴S¹⁶O⁺ at 34→50. ⁶³Cu was determined at 63→63, and ⁶⁵Cu was determined at 65→65. For the purposes of this experiment, sulfur was detected at 34→50 and copper was detected at 63→63.

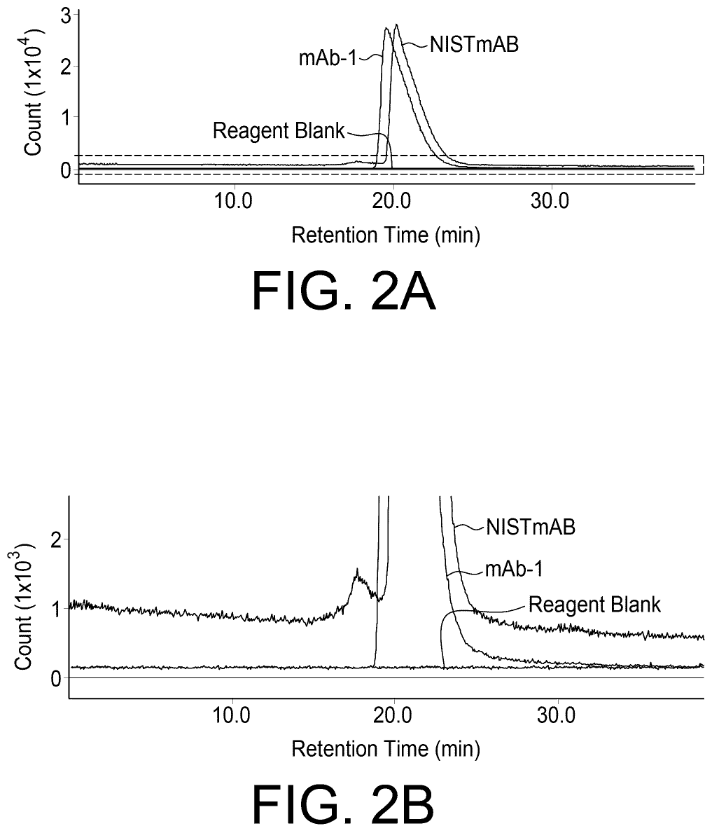

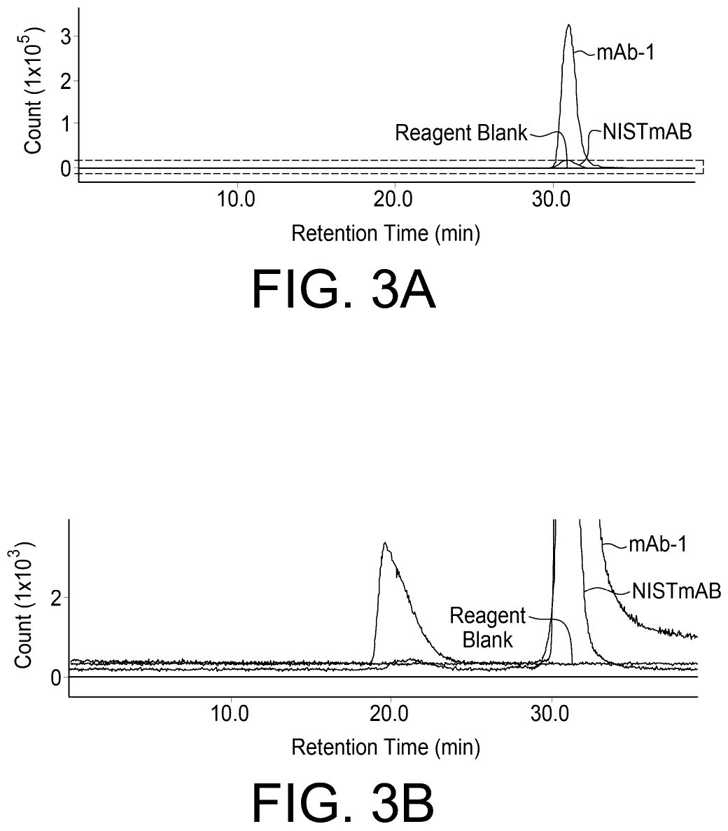

The LC-ICP-MS method was used to successfully separate signals from copper bound to mAb-1 and copper in excipients. Each was identified by comparing an LC-ICP-MS chromatogram of sulfur (FIG. 2A, zoomed in version shown in FIG. 2B) to an LC-ICP-MS chromatogram of copper (FIG. 3A, zoomed in version shown in FIG. 3B). As shown in FIG. 2A, sulfur was used to detect a monomer protein signal at around 20 minutes for mAb-1, and around 21 minutes for NISTmAb. A high molecular weight (HMW) species was detected at around 18 minutes, which can be more easily viewed in the zoomed in version of the sulfur chromatogram shown in FIG. 2B. The mAb-1 monomer peak detected at 20 minutes in the sulfur chromatogram therefore confirmed the identify of the peak eluting at around 20 minutes in the copper chromatogram (FIG. 3A) as copper bound to mAb-1. The peak corresponding to copper bound to mAb-1 can also be more easily viewed in the zoomed in version of the copper chromatogram shown in FIG. 3B. The peak eluting at around 30 minutes in the copper chromatogram (FIG. 3A) was therefore identified as copper in the excipients. Further, significantly higher amounts of copper were observed in mAb-1 and its excipients as compared to the standard NISTmAb.

Therefore, the LC-ICP-MS method and system was useful for separation and analysis of metals bound to mAbs, metals bound to small molecules, and free metals in a mAb sample. However, the LC-ICP-MS method was limited for some applications because proteins are monitored by sulfur, which can only be analyzed under O₂ mode.

### Example 2: Development of LC-UV-ICP-MS Methods for Metal-mAb Analysis

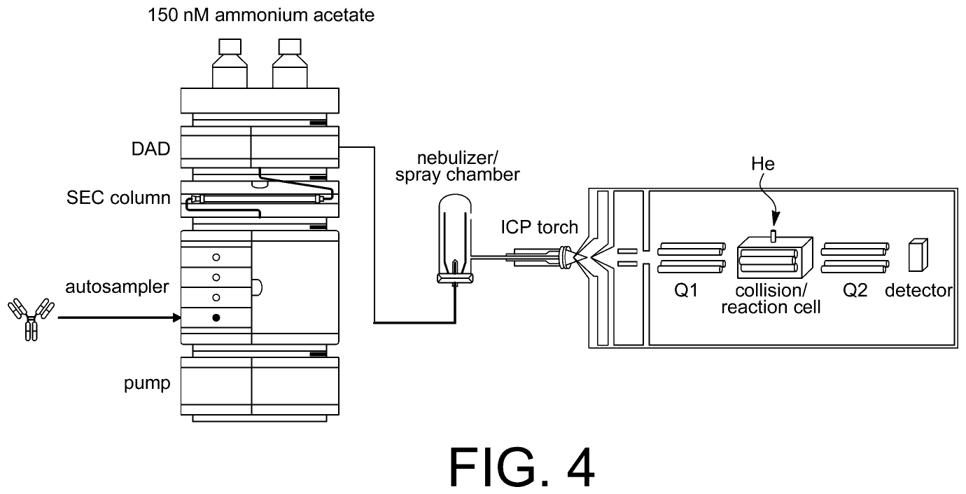

In order to characterize multiple metals in a mAb sample, the LC-UV-ICP-MS methods and systems described above were modified. As depicted in FIG. 4, the ICP-MS was operated in helium (He) collision mode to effectively and reliably remove multiple polyatomic interferences for most elements, and a diode-array detector (DAD) was used to monitor the ultraviolet (UV) signals of proteins.

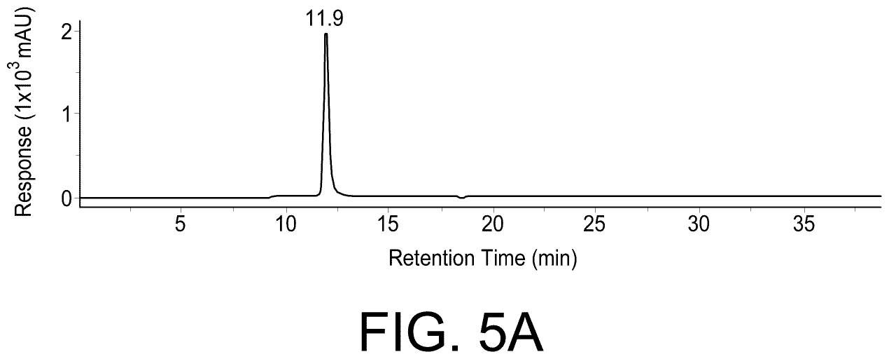

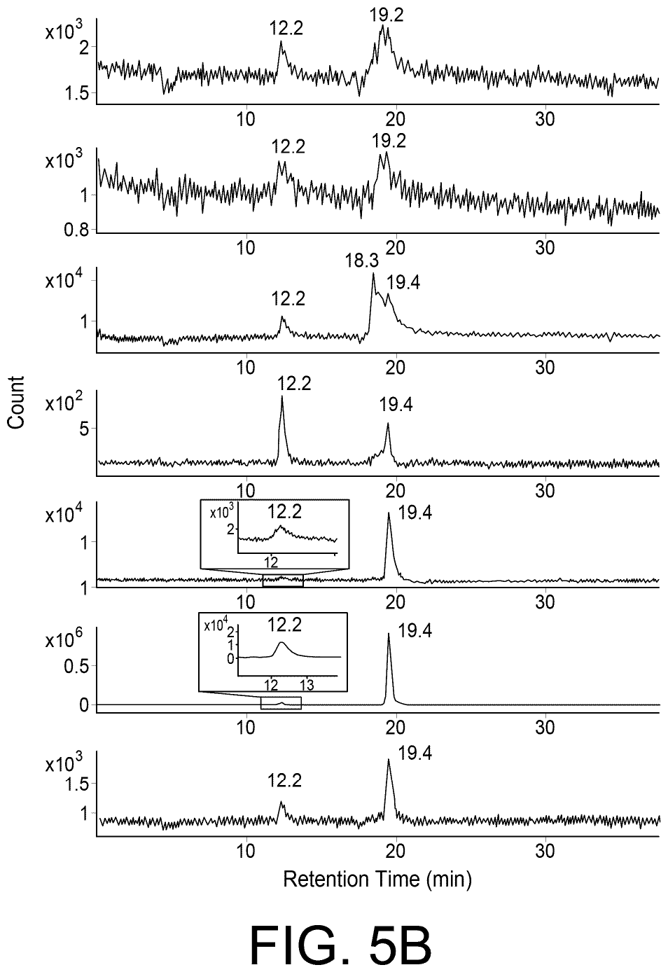

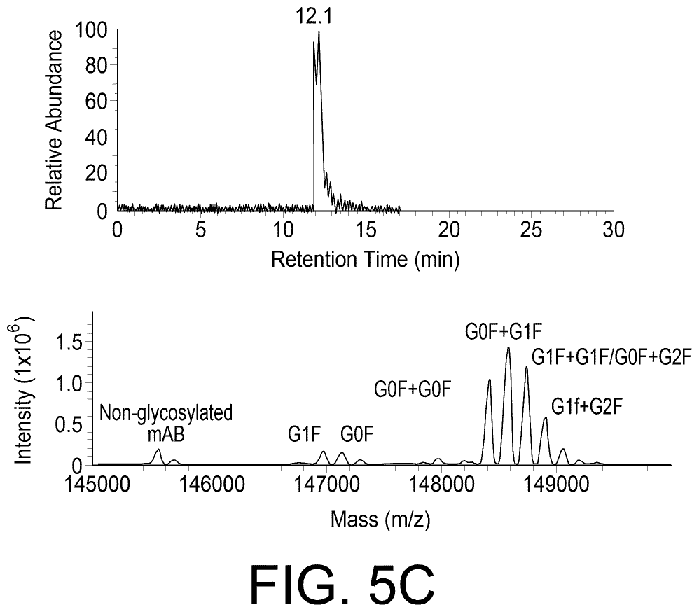

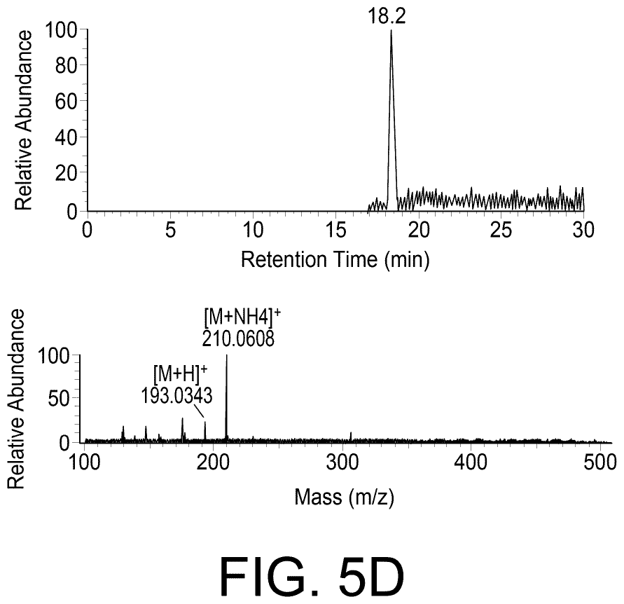

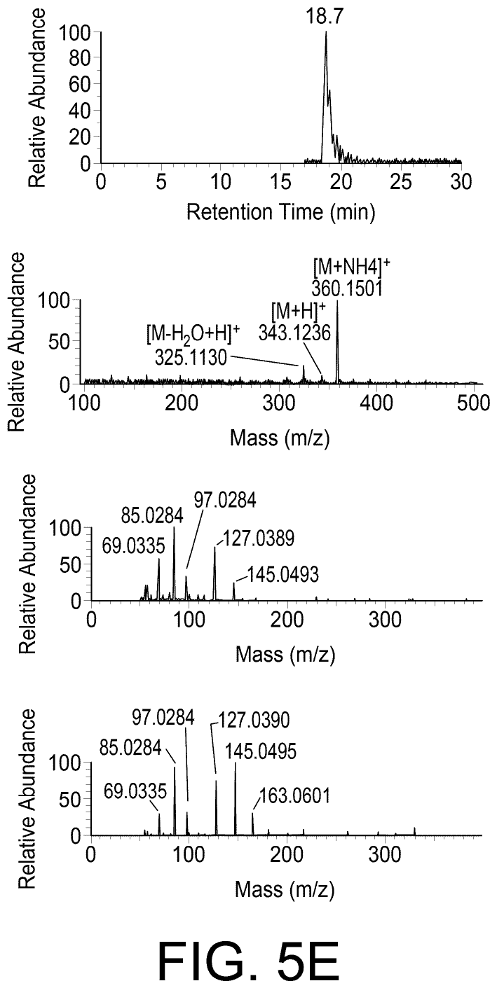

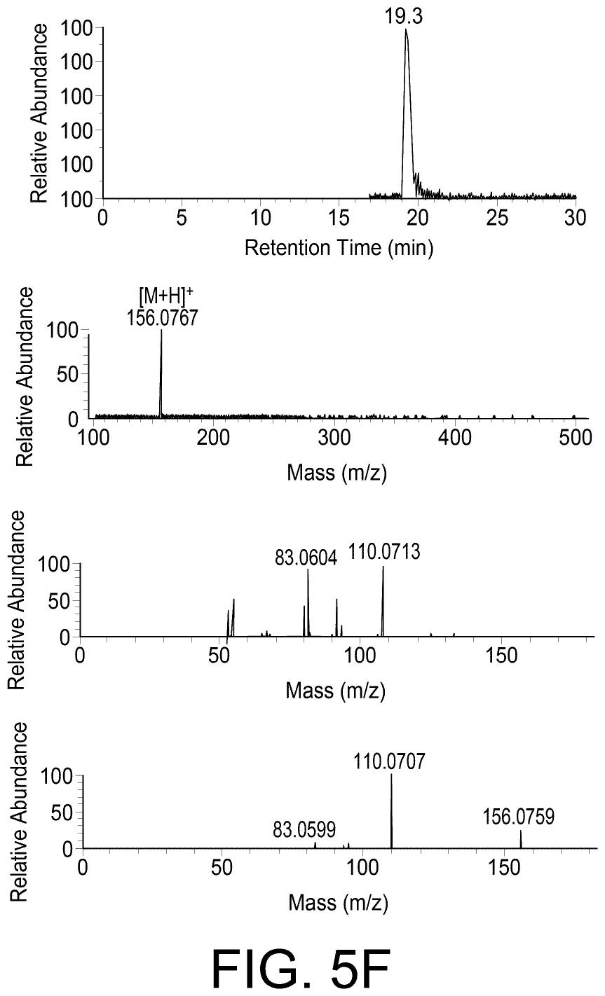

A final concentrated pool of a monoclonal antibody, mAb-1, was analyzed using the LC-UV-ICP-MS method for the presence of 12 trace metals. A total of 100 μg of the mAb-1 sample was loaded on the column to avoid the saturation of the UV detector and ensure the detection of the metals with low intensity signals. Ultraviolet detection at an absorbance of 280 nm identified a peak at about 11.9 minutes (FIG. 5A). ICP-MS detected seven metals (Cr, Mn, Fe, Co, Ni, Cu, and Zn) bound to mAb-1 (FIG. 5B). No interaction was detected between Mg, Al, Ti, V, Mo and mAb-1 (data not shown). For the seven metals, peaks were detected around 19 min on the ICP-MS chromatograms, but no protein signal was identified on the UV chromatogram. This suggests that the peaks on the ICP-MS chromatograms represent interactions between metals and small molecules within the solution. Notably, multiple peaks between 18.3 min and 19.4 min were observed on the ICP-MS chromatogram for iron, which suggests that iron interacts with more than one small molecule.

A base peak chromatogram of full scan with 2500-6000 m/z of mAb-1 identified a peak at about 12.1 minutes, with corresponding deconvoluted mass spectra, as shown in FIG. 5C. Three small molecules (citrate, sucrose, and histidine) were detected. For citrate, an extracted ion chromatogram (EIC) at 210.0608 m/z (top panel) and MSI spectra (bottom panel) are shown in FIG. 5D. For sucrose, an EIC at 360.1501 m/z (top panel), MS1 spectra (second panel), observed MS2 spectra at 360.1501 m/z (third panel), and in-house database MS2 spectra for sucrose (bottom panel) are shown in FIG. 5E. For histidine, an EIC at 156.0767 m/z (top panel), MS1 spectra (second panel), observed MS2 spectra at 156.0767 m/z (third panel), and in-house database MS2 spectra for histidine (bottom panel) are shown in FIG. 5F.

These results indicate that LC-UV-ICP-MS methods and systems were useful for identifying components of mAb samples (including mAb size variants) that bound to a variety of metal species. However, the LC-UV-ICP-MS methods were limited because they did not simultaneously provide structural identification of metal-binding molecules.

### Example 3: Development of LC-UV-ICP-MS-HRMS Methods for Metal-mAb Analysis

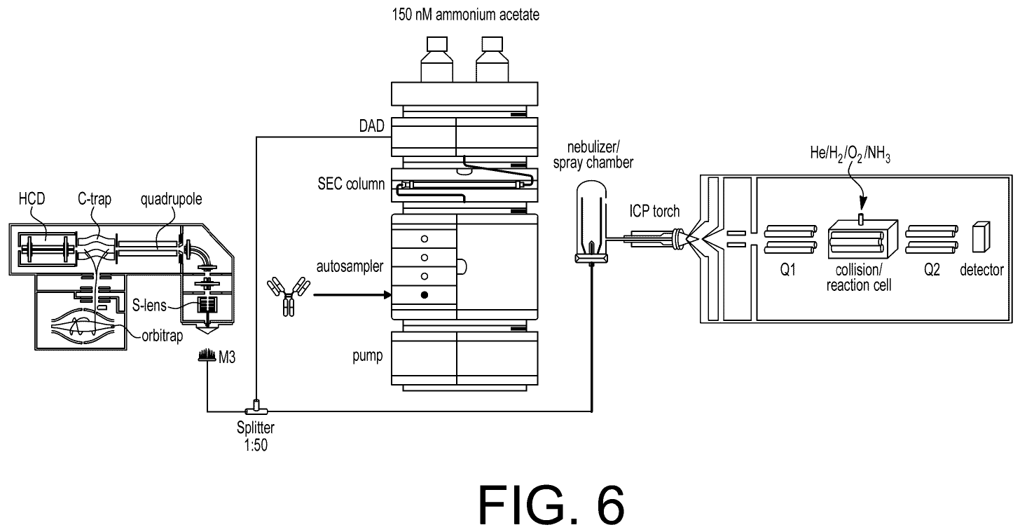

In order to provide structural identification of protein or polypeptide and small molecule species associated with elements in a sample, for example metals, while simultaneously characterizing the elements, novel LC-UV-ICP-MS-HRMS methods and systems were developed. Native SEC (nSEC)-UV was simultaneously coupled with ICP-MS for elemental analysis, for example, metal analysis, and a Thermo ORBITRAP EXPLORIS™ 240 Mass Spectrometer with NEWOMICS® microflow-nanospray electrospray ionization (MnESI) source for structural identification of element-binding molecules, for example metal-binding molecules, as shown in FIG. 6. The MnESI source was equipped with a M3 emitter, and the ICP-MS was operated in helium (He) collision mode.

Given that metals in mAb samples are distributed across both proteins and small molecules, it was necessary to obtain HRMS data at both high m/z and low m/z ranges to identify metal-bound proteins and small molecules. Conducting these acquisitions in separate injections could result in variations in retention time, thereby increasing the complexity of identification when comparing the profiles of nSEC-UV, ICP-MS and MS chromatograms. To address this, the HRMS acquisition was streamlined by initially performing a full scan range of either 1000-60000 m/z or 2500-6000 m/z in the protein elution region, and then switching to a low mass range with a full MS scan of either 60-1000 m/z or 100-500 m/z followed by data-dependent acquisition in the small molecule region.

The LC-UV-ICP-MS-HRMS platform allowed for analysis of element distribution over different forms (e.g., metal bound to monomeric mAb, HMW species, LMW species, small molecules, etc.) under native conditions. nSEC-UV, ICP-MS, and HRMS data could be acquired in one injection, enabling detection and identification of element-bound molecules, for example metal-bound molecules, simultaneously.

As depicted in FIG. 6, a three-way flow splitter was used to connect the LC-UV system, ICP-MS system and HRMS system. A flow of 200 μL/minute from the LC system was split 1:20, with 10 μL/minute flowing to the NEWOMICS® MnESI source of the HRMS system and 190 μL/minute flowing to the ICP-MS system.

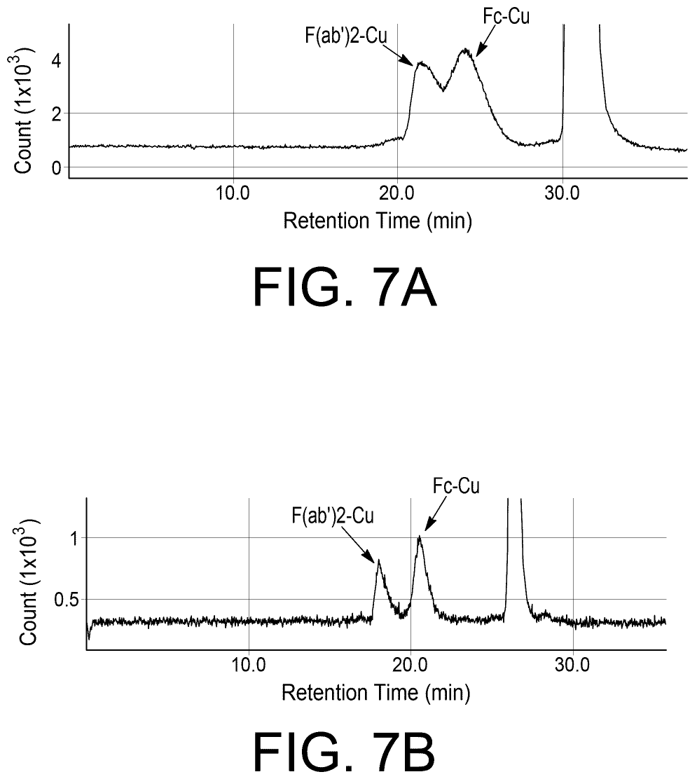

The tubing for the LC-UV-ICP-MS-HRMS system was modified to improve separation of different species. An initial tubing used to connect the splitter to the ICP-MS, via a MicroMist nebulizer, had a tubing size of 0.25 mM internal diameter (I.D.), with a flow rate of 0.18 mL/minute. This tubing led to an overlap in peaks of a F(ab′)2-Cu species and a Fc-Cu species, as shown in FIG. 7A. Additional modifications led to the selection of a quartz concentric nebulizer, which allowed for the use of tubing with 0.13 mM I.D. at a flow rate of 0.2 mL/minute, and led to good separation of the two peaks, as shown in FIG. 7B.

The LC-UV-ICP-MS-HRMS method was applied for analysis of monoclonal antibodies mAb-2 and mAb-3. An amount of 400 μg of a final concentrated pool of mAb-2 or mAb-3 was injected into the system in helium mode.

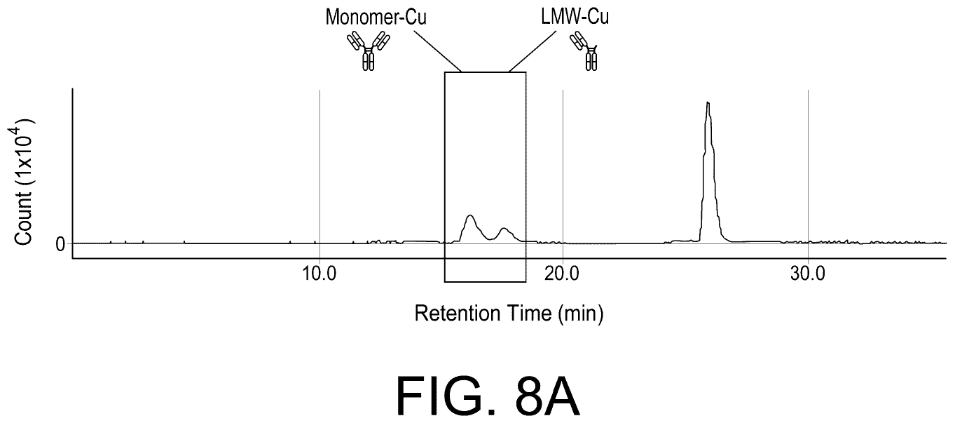

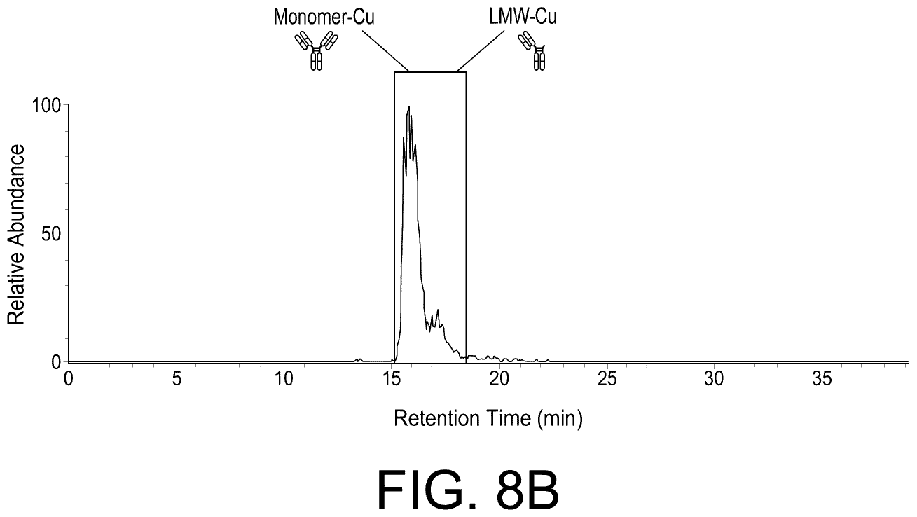

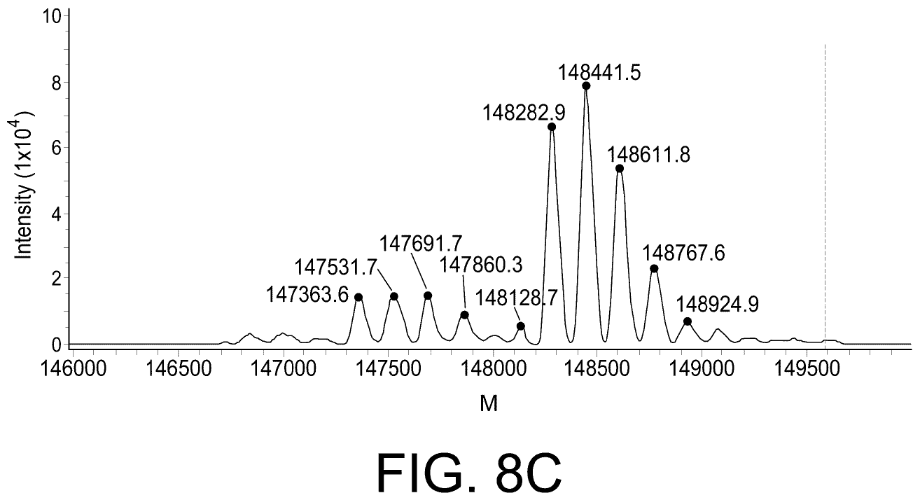

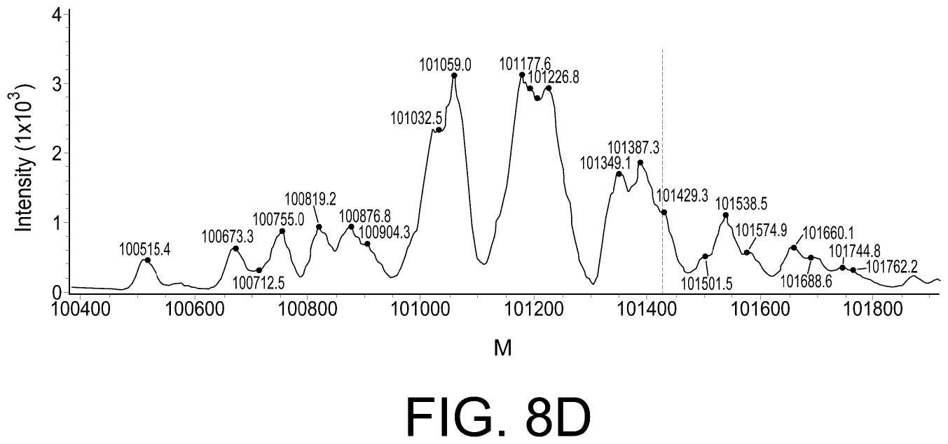

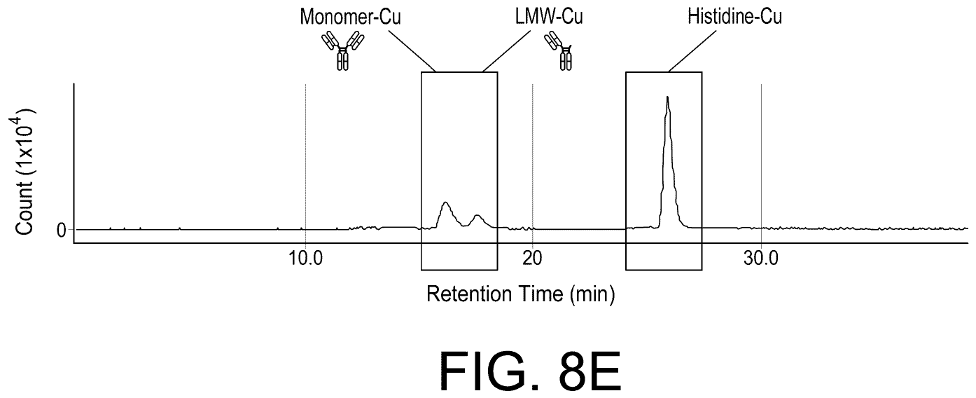

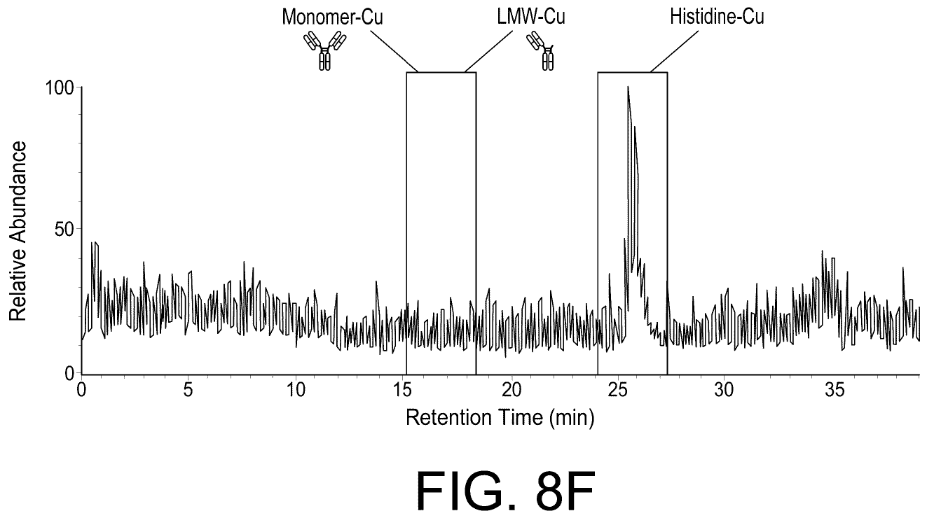

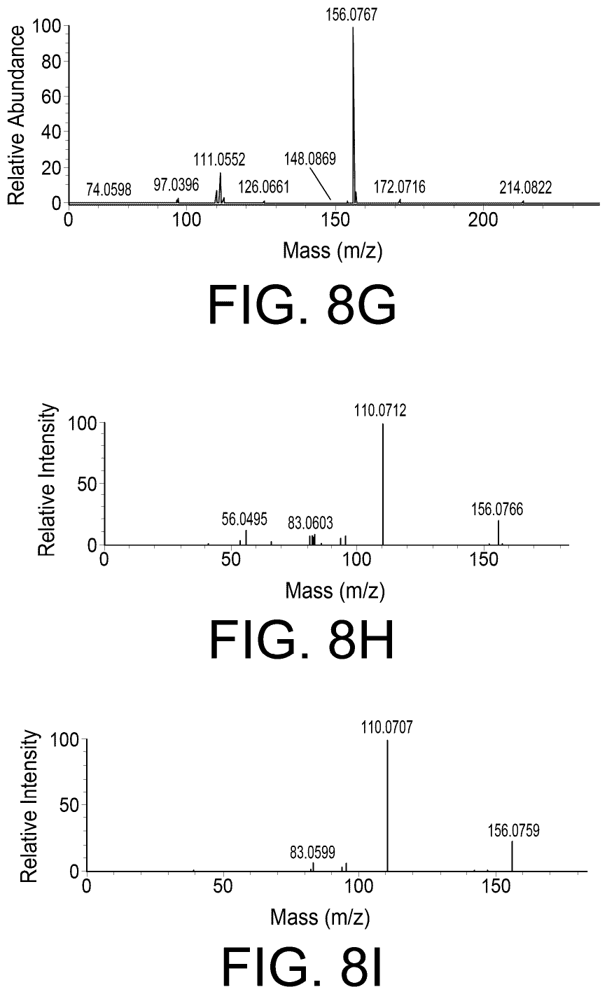

As shown in the ICP-MS copper chromatogram of FIG. 8A, copper bound to mAb-2 monomer (“monomer-Cu”) and low molecular weight species (“LMW-Cu”) were detected as separate peaks eluting at around 16 minutes and 18 minutes, respectively. A full scan of 1000-6000 m/z confirmed the identities of the peaks as mAb-2 monomer-Cu and LMW-Cu, as shown in the TIC mass spectrum of FIG. 8B. The peaks were further confirmed with deconvoluted mass spectra for the monomer-Cu species (FIG. 8C) and the LMW-Cu species (FIG. 8D). For the LMW-Cu species, the most abundant masses were identified as half mAb-2, plus a truncated heavy chain with Cys/Asp clipping. As shown in the ICP-MS chromatogram of FIG. 8E, the full scan of 1000-6000 m/z also detected copper bound to histidine (“histidine-Cu”) in the mAb-2 sample. The identity of the peak as copper bound to histidine was confirmed by an MS1 spectrum at full scan of 60-1000 m/z (FIG. 8F), a data-dependent MS/MS (ddMS2) spectrum at 156.0767 (FIG. 8G), which were matched to a library reference mass spectrum for histidine (FIG. 8H). Thus, monomer species, LMW species, and the small molecule histidine were detected in the mAb-2 sample.

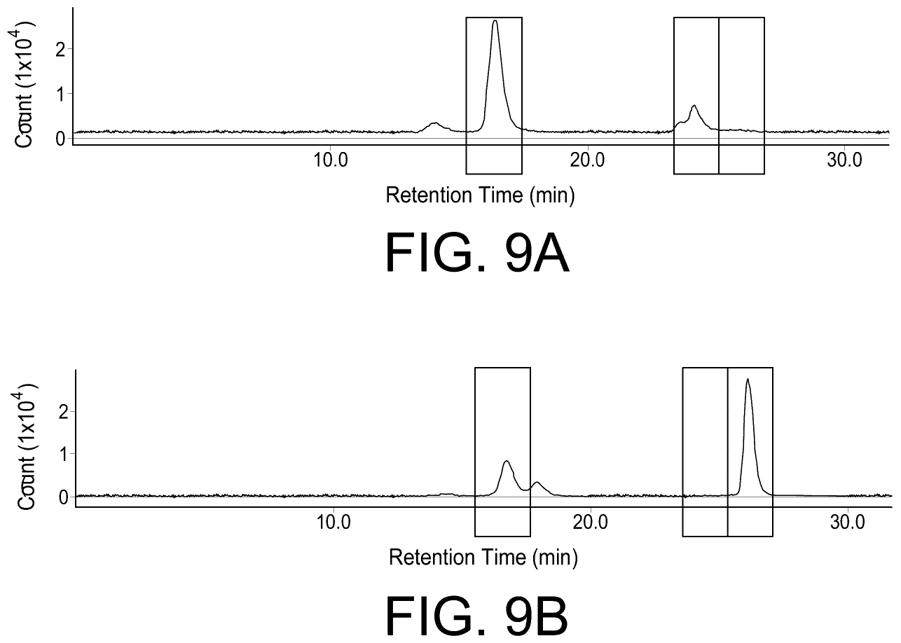

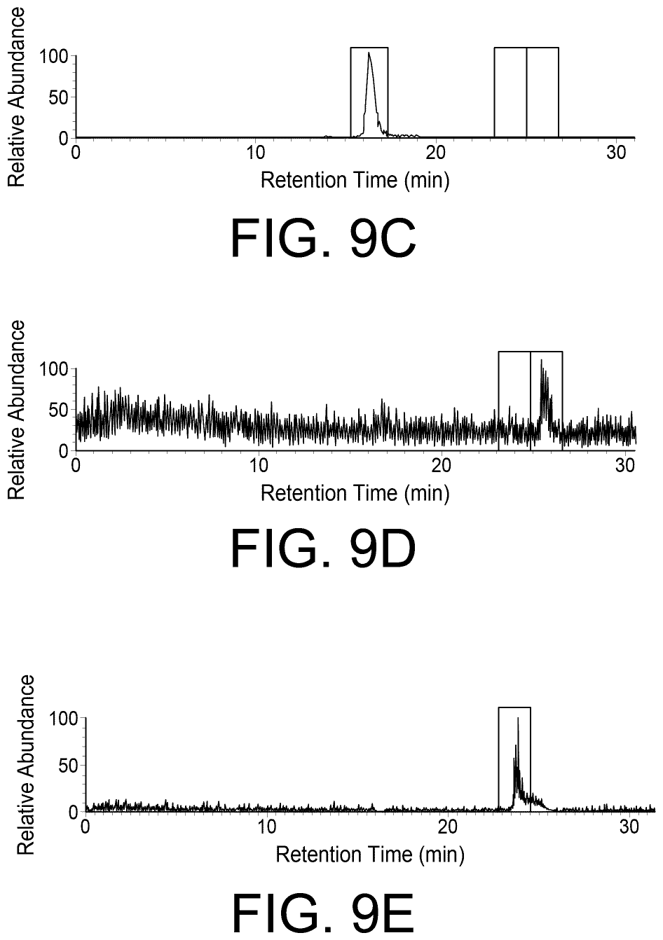

The LC-UV-ICP-MS-HRMS method was applied to detect both iron (Fe) and copper (Cu) for the mAb-3 sample. As shown in the ICP-MS iron chromatogram of FIG. 9A, iron bound to mAb-3 monomer (“monomer-Fe”), high molecular weight species (“HMW-Fe”), and citrate (“citrate-Fe”) were detected as separate peaks eluting at around 17 minutes, around 15 minutes, and around 25 minutes, respectively. As shown in the ICP-MS copper chromatogram of FIG. 9B, copper bound to mAb-3 monomer (“monomer-Cu”), low molecular weight species (“LMW-Cu”), and histidine (“histidine-Cu”) were detected as separate peaks eluting at around 17 minutes, around 18 minutes, and around 27 minutes, respectively. A full scan of 1000-6000 m/z shown in the base peak mass spectrum of FIG. 9C confirmed the identity of the mAb-3 monomer at around 17 minutes. A full scan of 60-1000 m/z shown in the base peak mass spectrum of FIG. 9D confirmed the presence of the peak corresponding to histidine-Cu species, and an extracted ion chromatogram at 210.0608 m/z confirmed the presence of the peak corresponding citrate-Fe species. The identity of the peak corresponding to the monomer species was further confirmed by the deconvoluted mass spectrum (data not shown), and the peaks corresponding to citrate and histidine were further confirmed by MS1 spectra of citrate and histidine (data not shown). Thus, the monomer species, LMW species, HMW species, and the small molecules citrate and histidine were detected in the mAb-3 sample.

The acquired HRMS data not only confirmed the identity of mAb-2 and mAb-3, but also facilitated the structural identification of various small molecules by matching MS1 peak and/or MS2 spectra to an in-house library of MS/MS data. Interestingly, three small molecules, citrate, sucrose and histidine, were identified by the HRMS data, and their retention times corresponded with the peaks of iron or copper bound small molecules observed on the ICP-MS chromatograms. Sucrose and histidine are commonly used excipients in the mAb formulation buffer. Low amount of citrate may remain as an impurity in mAb samples and may originate from buffer solutions utilized during the downstream purification process. Indeed, citrate, sucrose, and histidine are all known to have an affinity for iron and copper. This affinity allows the small molecules to bind to iron and copper, which could explain the observed peaks on the ICP-MS chromatograms.

The SEC column, which had a pore size of 200 Å, was specifically designed to separate proteins and their aggregates with molecular weights ranging from 10 k to 450 k Da. Surprisingly, rather than elute together, the three small molecules of citrate, sucrose, and histidine separated using this SEC column. Differences in the retention times of the retention times could potentially have been due to secondary interaction between the analyte and the column particles. Further, citrate can form diverse coordination complexes with metal ions in aqueous solution, some of which may possess significantly larger molecular weights than sucrose and histidine

### Example 4: Identification of Copper Adducts in Truncated IgG1

The LC-UV-ICP-MS-HRMS methods and systems of the present disclosure were used to characterize interactions with copper, a variety of IgG1 monoclonal antibodies, and a variety of IgG4 monoclonal antibodies. In particular, the ability to detect copper bound to low molecular weight (LMW) species of these antibodies allowed for the characterization of the LMW species as being-or not being-copper adducts. The following IgG1 antibodies were tested: NISTmAb, mAb-8, mAb-5, mAb-6, mAb-3, and mAb-2. The following IgG4 antibodies were tested: mAb-1 and mAB-7.

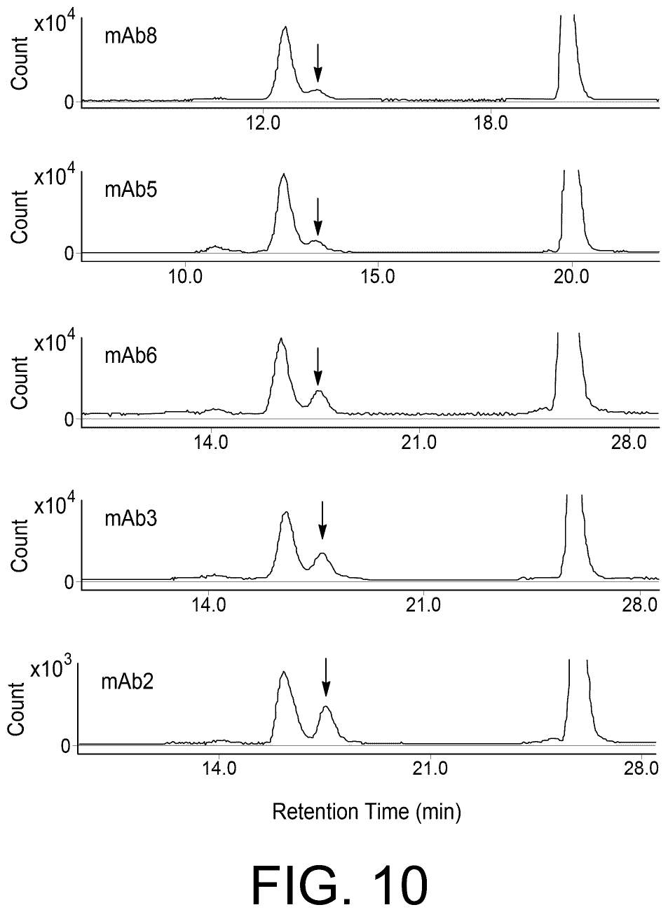

As shown in FIG. 10, nSEC-IP-MS copper chromatograms were generated for each of IgG1 antibodies mAb-8, mAb-5, mAb-6, mAb-3, and mAb-2. In each chromatogram, copper bound to monomer was detected as a separate peak from copper bound to a low molecular weight (LMW) species. In FIG. 10, the LMW-Cu peak is indicated by an arrow for each chromatogram.

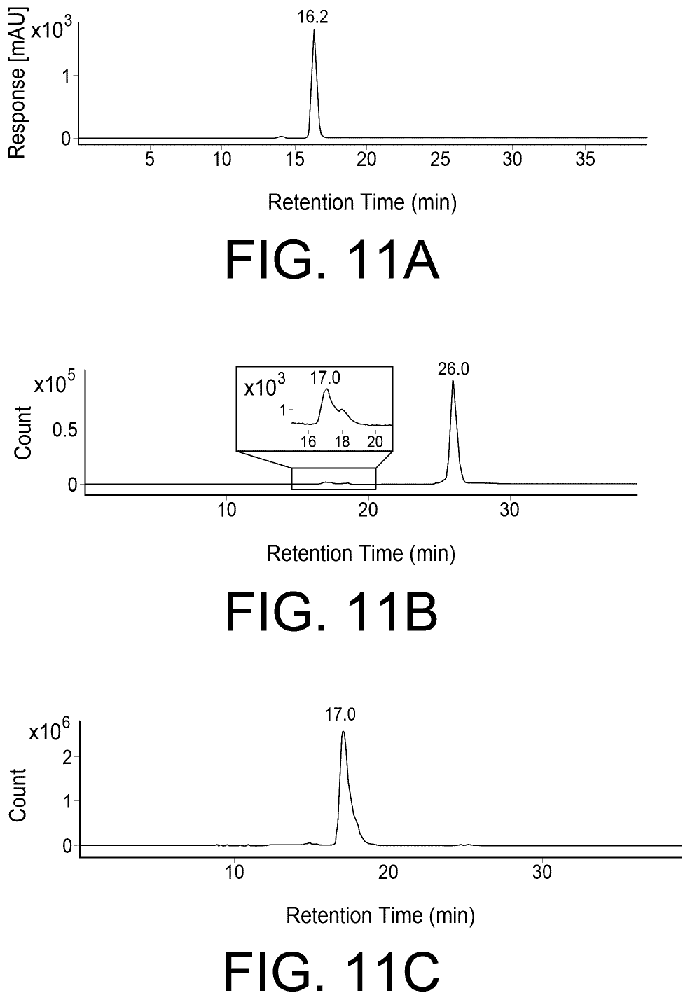

As shown in FIGS. 11A-11C, copper and sulfur were used to detect an LMW species of NISTmAb, eluting at around 17 minutes. An nSEC-UV chromatogram for NISTmAb is shown in FIG. 11A. An nSEC-ICP-MS copper chromatogram is shown in FIG. 11B. The corresponding LMW peak on the nSEC-UV chromatogram (FIG. 11A) eluted earlier than that the corresponding LMW peak on the nSEC-ICP-MS copper chromatogram (FIG. 11B), because the liquid chromatography eluate reached the UV detector before reaching the ICP-MS detector. This discrepancy was not observed in an nSEC-ICP-MS sulfur chromatogram, shown in FIG. 11C, indicating that sulfur could be used to monitor protein signal more effectively than copper.

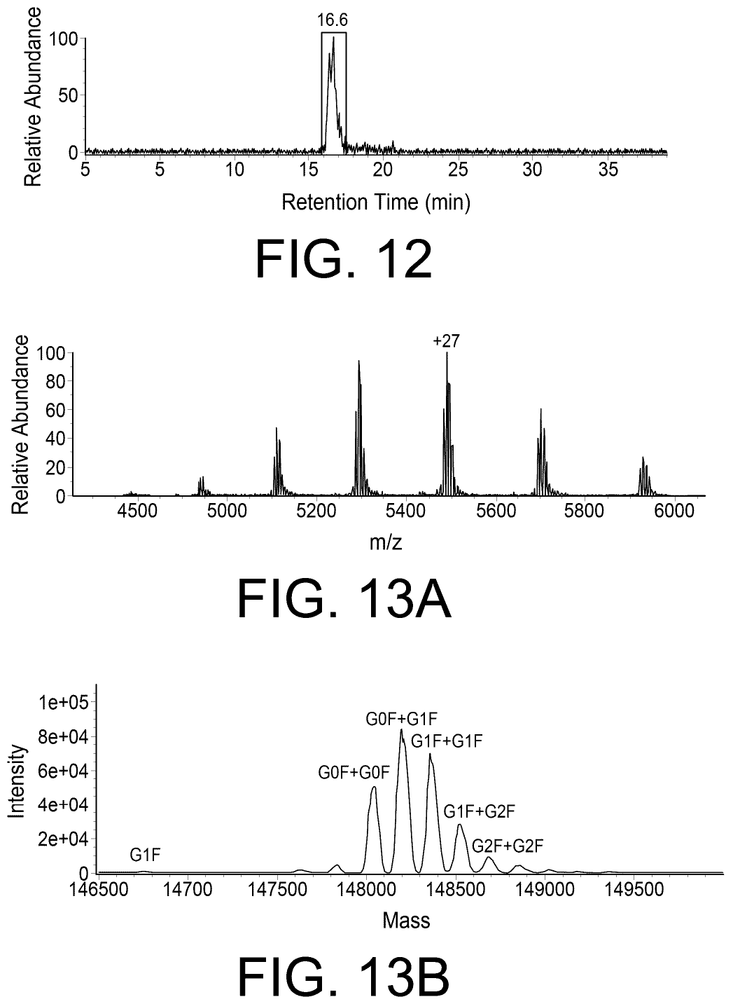

As shown in FIG. 12, a total ion chromatogram at full scan 2500-6000 m/z showed a peak eluting at a similar retention time as the peak corresponding to NISTmAb observed in the nSEC-UV chromatogram (FIG. 11A). As shown in FIG. 13A, the averaged raw mass spectra from this peak displayed symmetric m/z peaks and high signal-to-noise ratios, indicating that the MnESI-HRMS settings allowed for good spectrum quality under the selected split flow from LC. As shown in FIG. 13B, the deconvoluted mass spectra identified different glycoforms and confirmed the identity of NISTmAb.

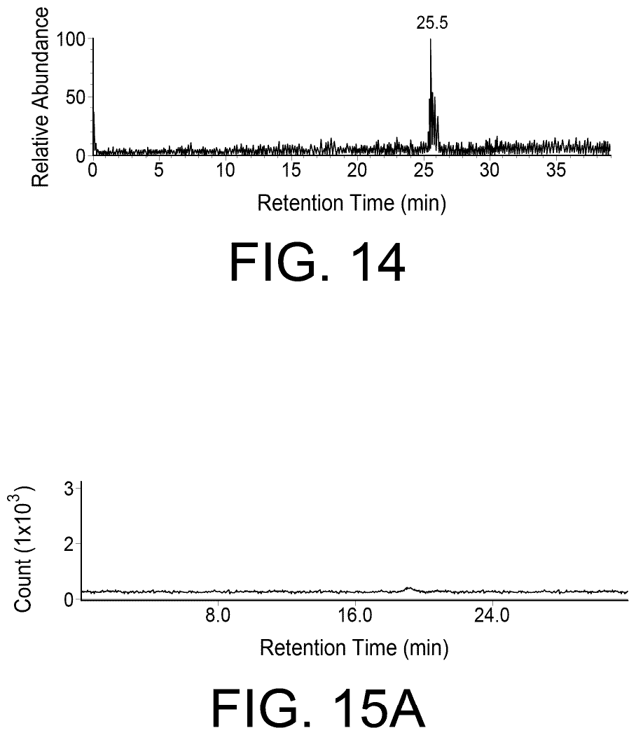

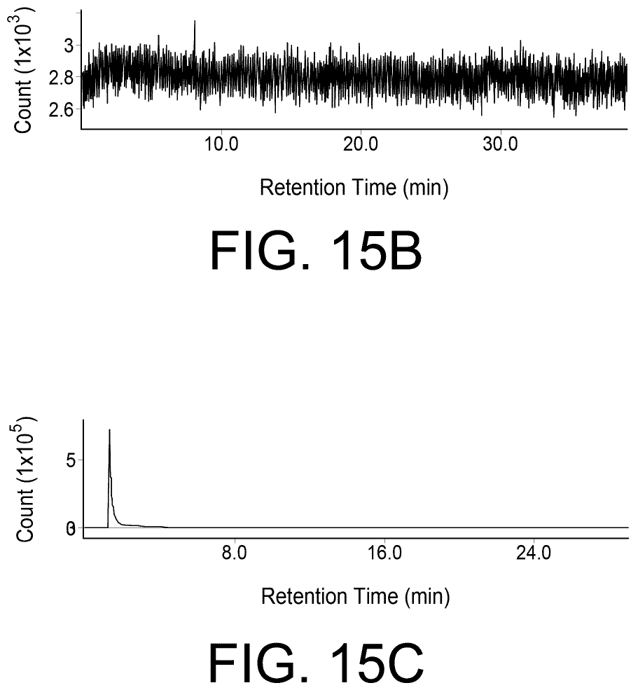

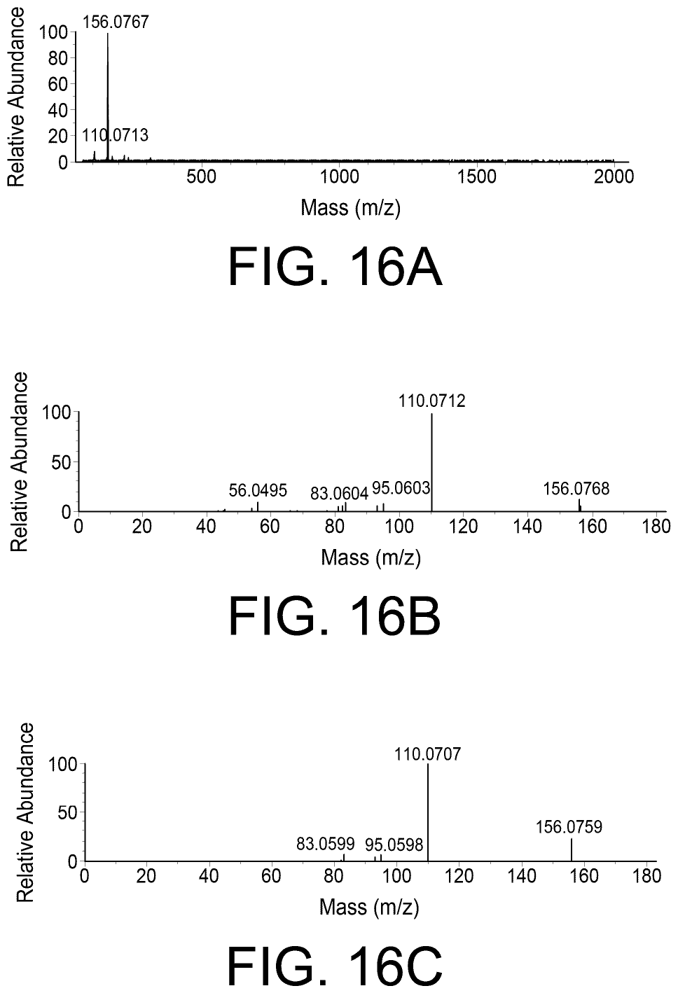

As shown in FIG. 14, a base peak chromatogram at full scan 60-2000 m/z showed a peak eluting at a similar retention time (around 26 minutes) as a later peak observed in the nSEC-ICP-MS copper chromatogram (FIG. 11B), which did not correspond to a protein signal. To determine whether this peak was a free copper salt or copper bound to small molecules, a 0.6 μM Cu (II) standard was injected onto the SEC column. Surprisingly, no free copper signal was detected (FIG. 15A). This was also the case for other metal salts, such as 4 mM Fe (III) standard injected onto the SEC column (FIG. 15B). However, free iron salt was detectable at the solvent front when injecting a 4 mM Fe (III) onto a strong anion exchange (SAX) column (FIG. 15C). These findings indicated that free metals may have been absorbed by the SEC column, and therefore, were undetectable by SEC. HRMS analysis of the peak at 26 minutes included producing an MS1 spectrum (FIG. 16A) and an MS spectrum of 156.0767 m/z (FIG. 16B), which were compared to a known MS2 reference spectrum for histidine (FIG. 16C). This analysis indicated that the peak at 26 minutes corresponded to copper-bound histidine.

The LMW species had previously been identified in various IgG1 molecules by native SEC-MS analysis. Modifications at clipping sites include addition of C₂O₃ at cysteine-aspartate clipping, addition of CO—H₂ at cysteine-aspartate clipping, and a lysine-threonine clipped species with an additional +63 Da mass corresponding to a copper adduct. The results from LC-UV-ICP-MS-HRMS analysis provided further evidence that the previously identified LMW +63 Da species of these IgG1 antibodies were copper adducts.

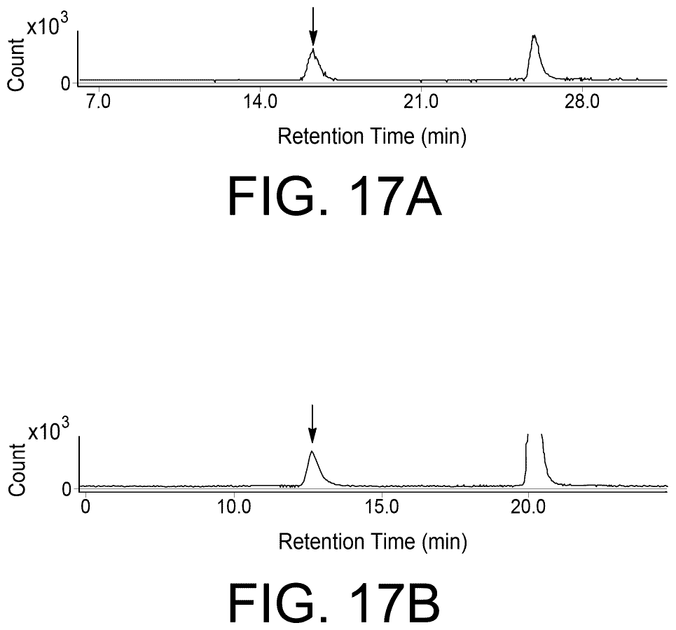

Unlike IgG1 antibodies, IgG4 antibodies do not have a copper binding pocket with a peptide sequence of DKTH at the hinge region. See, e.g., Wenig et al., “Structure of the streptococcal endopeptidase IdeS, a cysteine proteinase with strict specificity for IgG”, PNAS USA, 2004, volume 101, pages 17371-17376. HRMS analysis was carried out for IgG4 antibodies mAb-1 and mAb-7. ICP-MS chromatograms for both mAb-1 (FIG. 17A) and mAb-7 (FIG. 17B) each showed a peak corresponding to a monomer-Cu species (identified by arrow), but did not show a peak corresponding to an LMW-Cu species. Thus, low molecular weight species of the IgG4 antibodies did not sufficiently bind copper so as to allow for detection, providing evidence that the LMW species of these IgG4 antibodies were not copper adducts.

### Example 5: Investigation of Metal-Based Coloration in mAb Products

The methods of the present disclosure were applied to an investigation of an abnormal color in a protein product. A final concentrated (213 mg/mL) pool of IgG1 monoclonal antibody mAb-3 was observed to have a dark color, as compared to water. A dark color was also observed in a protein A chromatography pool of the same mAb-3. Two approaches were used to investigate the source or case of the observed dark color. First, the mAb-3 sample and a mAb-2 sample were directly injected into an ICP-MS system to identify any differential metals between the two samples. Second, multiple types of analyses were performed, including analyzing intact mAb, IdeS-digested mAb (subunit analysis), EDTA-incubated mAb, and denatured mAb.

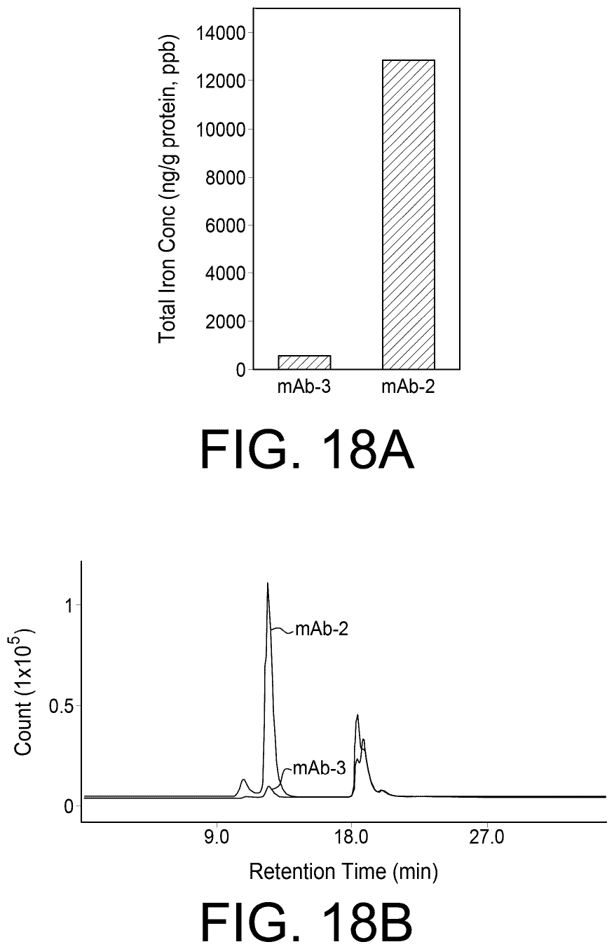

As shown in FIG. 18A, direct injection ICP-MS results showed that the total iron concentration in the mAb-2 sample was greater than 20 times as much as the concentration in the mAb-3 sample. To determine whether the increased iron in the mAb-2 sample was bound to the antibody or derived from solution, an iron speciation analysis was carried out using nSEC-UV-ICP-MS-HRMS. As shown in FIG. 18B, the nSEC-ICP-MS chromatograms showed that the amount of iron bound to monomer and high molecular weight (HMW) species of mAb-2 (eluting at around 11 and 13 minutes, respectively) was 20 times greater than iron bound to monomer and HMW species of mAb-3. The amount of iron bound to small molecules (eluting at around 19 minutes) was quite similar between mAb-2 and mAb-3. Thus, the excess iron detected in the mAb-2 sample originated from iron bound to the antibody, rather than iron bound to small molecules in solution.

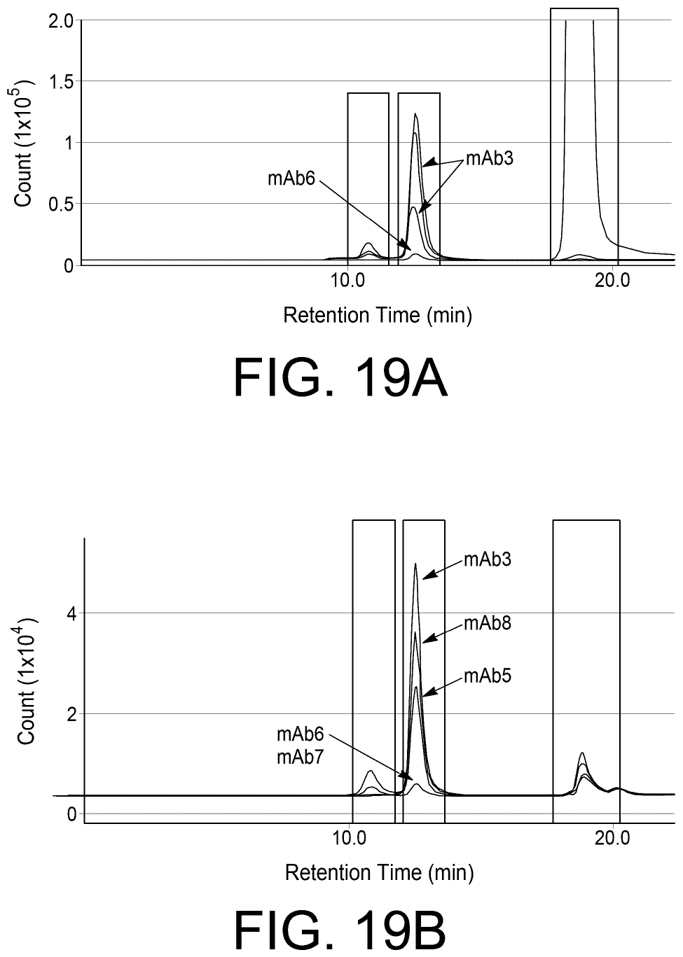

Additional analysis of mAb-3 samples from various stages of processing (including protein A chromatography eluate, HIC eluate, and AEX eluate) and samples of other mAbs (mAb-5, mAb-6, mAb-7, and mAb-8) were conducted using intact nSEC-ICP-MS-HRMS. Samples with undesirable coloration included mAb-3, mAb-5, and mAb-8, while samples that did not have undesirable coloration included mAb-6 and mAb-7. As shown in FIG. 19A and FIG. 19B, samples that had undesirable coloration (mAb-3, mAb-5, and mAb-8) had significantly higher amounts of iron bound to the antibodies, as compared to samples that did not have coloration (mAb-6 and mAb-7).

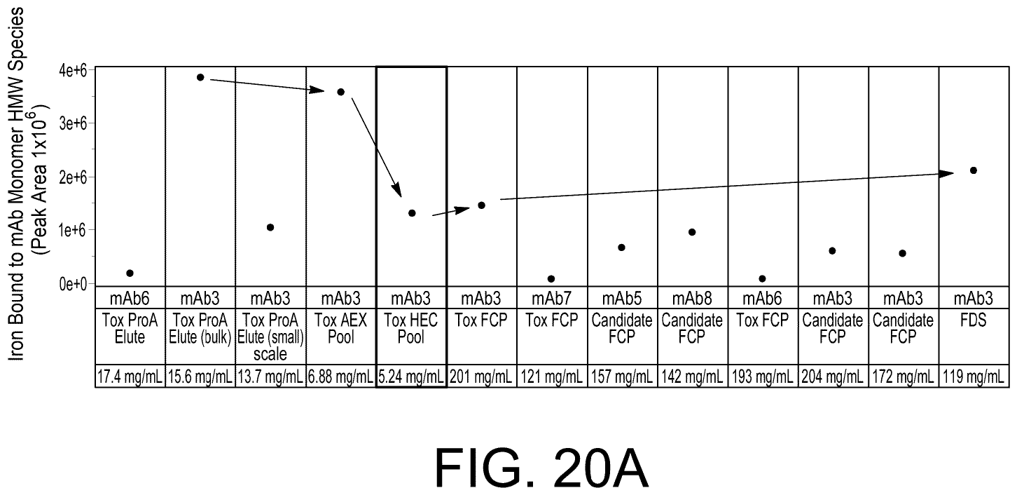

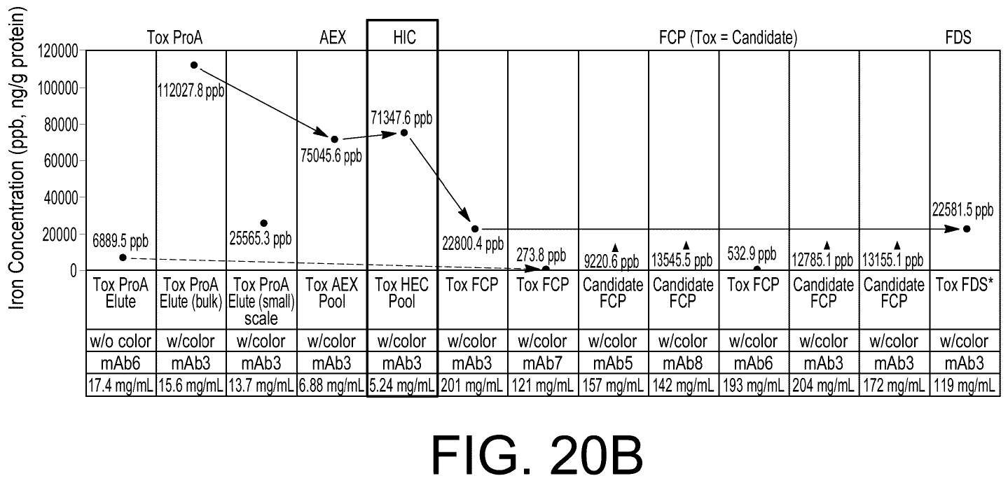

A comparison was also made between total levels of iron and mAb-bound iron across a variety of samples using the methods described above. Total iron was normalized to protein concentration. For analysis of mAb-bound iron, 100 μg of protein was loaded, and the peak area of HMW-Fe and monomer-Fe was quantified. As shown in FIG. 20A and FIG. 20B, the levels of mAb-Fe appeared to trend with the total concentration of iron in each sample, with the exception of the mAb-3 tox HIC pool, which contained a higher amount of citrate-Fe.

To determine if iron binding to mAb-2 was in a specific or unspecific manner, and to further characterize the location of the iron binding site, an IdeS digestion assisted nSEC-ICP-MS-HRMS analysis was performed with mAb-2 and mAb-3 samples. Of note, mAb-2 and mAb-3 differed only in the F(ab′)₂ region of their sequences. The antibody samples were digested into F(ab′)₂ and Fc fragments by incubating the sample with FABRICATOR® IdeS enzyme at a substrate: enzyme ratio of 1:1.25, at 37° C. for one hour, agitating at 650 rpm. IdeS was a cysteine protease that specifically targets IgG antibodies, cleaving them at a single amino acid site beneath the hinge region, yielding F(ab′)₂ and FC fragments while preserving native structures of the antibodies.

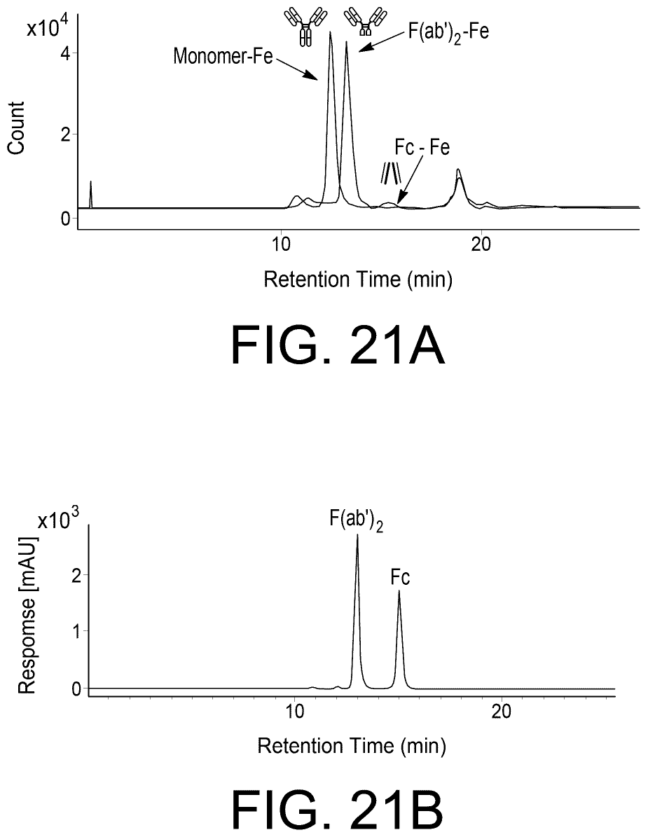

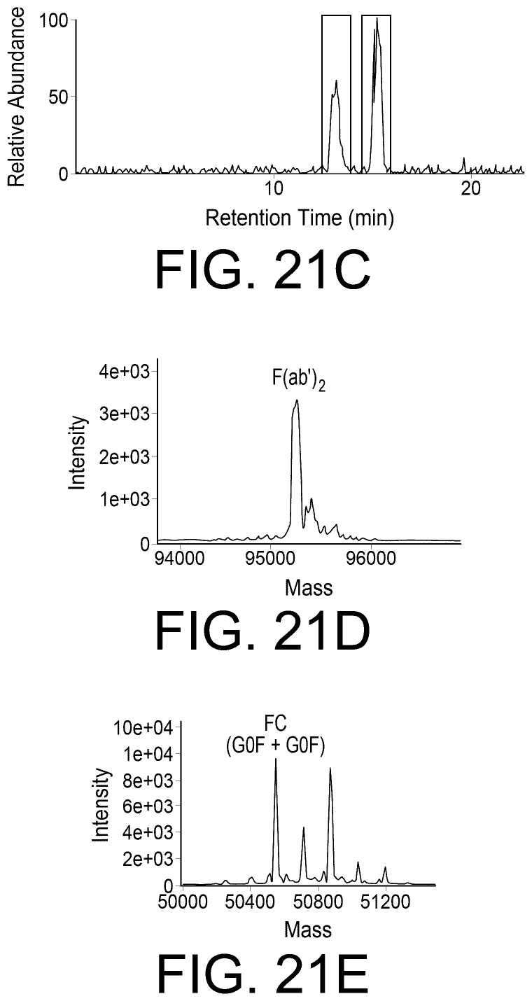

As shown in FIG. 21A, nSEC-ICP-MS-HRMS analysis showed that the F(ab′)₂ fragment of mAb-2 had significantly higher iron binding capacity (about 40 times more) than its Fc fragment. Indeed, the intensity of the peak for iron bound to the F(ab′)₂ fragment of mAb-2 was comparable to that of the peak for iron bound to the intact mAb-2. Similarly, a UV chromatogram shown in FIG. 21B confirmed that a greater amount of iron was detected as bound to the F(ab′)₂ fragment than to the Fc fragment of digested mAb-2. A total ion chromatogram at 2500-6000 m/z shown in FIG. 21C displayed the elution times of the F(ab′)₂ and Fc fragments of mAb-2 as being around 13 minutes and around 15 minutes, respectively. Deconvoluted mass spectra for the F(ab′)₂ peak (FIG. 21D) and the Fc peak (FIG. 21E) confirmed the identity of these peaks. These results indicated that the F(ab′)₂ region was the primary site of iron binding, accounting for the majority of iron binding to mAb-2.

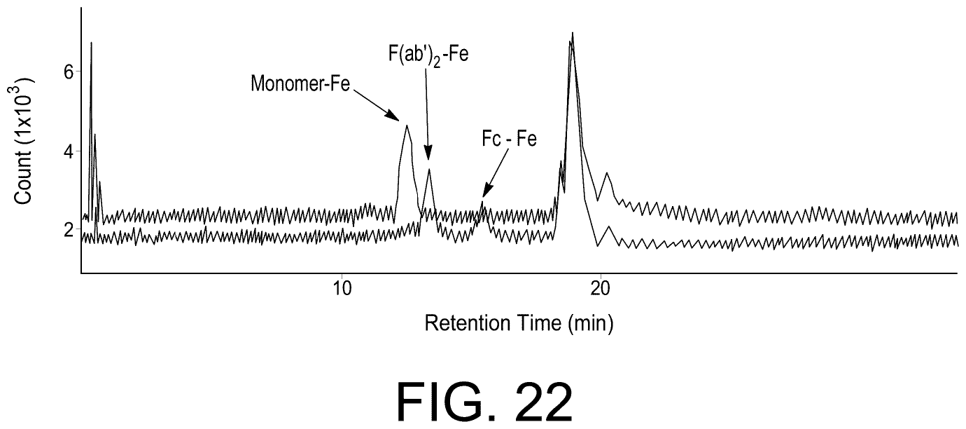

For mAb-3, the binding of iron to the F(ab′)₂ fragment was observed to be 1.7 times higher than its binding to its Fc fragment, as shown in the ICP-MS chromatogram shown in FIG. 22, but still significantly less than the F(ab′)₂-bound iron in mAb-2.

These results indicated that the excess iron in mAb-2 was primarily associated with the F(ab′)₂ subunit of the mAb-2 antibody. No iron signal was detected in the IdeS enzyme, indicating that the IdeS digestion process did not introduce any iron contamination. The LC-UV and HRMS data, acquired in the same injection as LC-ICP-MS, confirmed the identities of the F(ab′)₂ and F c fragments.

### Example 6: Treatment Conditions for Dissociating Metal-mAb Species

To investigate conditions for dissociating the metal-mAb complexes, samples having 10 mg/mL mAb-3 and 10 mg/mL mAb-6 were incubated with 50 mM of iron chelator EDTA at room temperature for one hour. The incubated samples with and without EDTA treatment were then analyzed by intact nSEC-ICP-MS-HRMS.

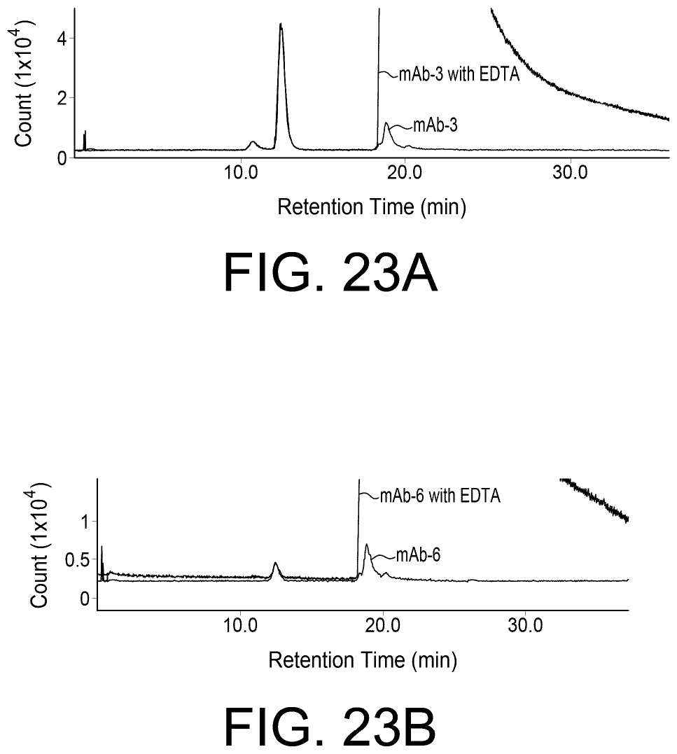

As shown in FIG. 23A, the iron chromatogram for the mAb-3 samples showed an HMW-Fe species eluting at around 11 minutes and the monomer-Fe species eluting at around 13 minutes for the mAb-3 samples with and without EDTA treatment, indicating that EDTA treatment did not remove monomer-Fe or HMW-Fe species. However, while the mAb-3 sample without EDTA treatment showed a small molecule-Fe peak eluting at around 19 minutes, the mAb-3 sample with EDTA treatment showed a significantly larger EDTA-Fe peak at this retention time, indicating that EDTA treatment removed small molecule-Fe species.

As shown in FIG. 23B, an iron chromatogram for the mAb-6 sample showed similar results. Samples of mAb-6 with and without EDTA treatment both showed a monomer-Fe species eluting at around 13 minutes. While the mAb-6 sample without EDTA treatment showed a small molecule-Fe species eluting at around 19 minutes, the mAb-6 sample with EDTA treatment showed significantly larger EDTA-Fe peak at this retention time, indicating that EDTA treatment removed small molecule-Fe species.

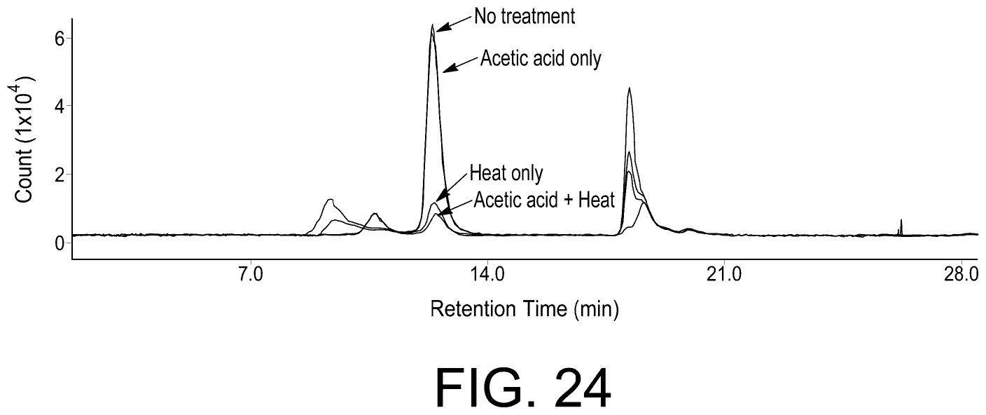

To investigate further potential treatment conditions, mAb-3 samples were additionally subjected to acid treatment (5 mM acetic acid incubation), heat treatment (incubation at 80° C. for 10 minutes while agitating at 800 rpm), or a combination of acid treatment and heat treatment, followed by intact nSEC-ICP-MS-HRMS analysis injecting 200 μg of mAb-3. As shown in FIG. 24, an iron chromatogram showed HMW-Fe species eluting between around 9-10 minutes for all samples, with monomer-Fe species eluting at around 12-13 minutes for all samples. Acid treatment had no effect on the formation of monomer-Fe species, but heat treatment did cause dissociation of monomer-Fe complexes.

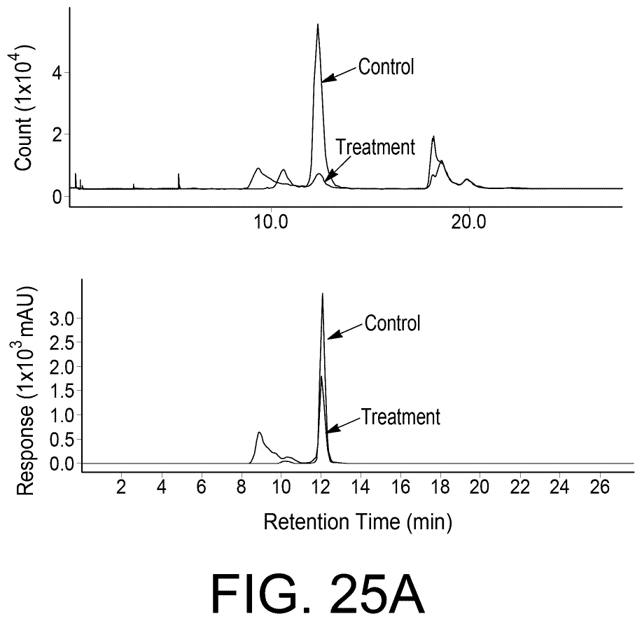

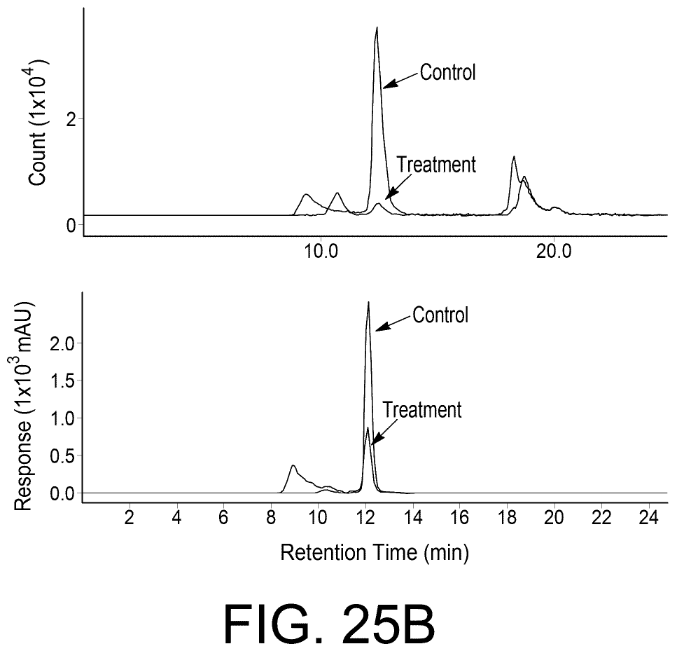

The effect of the combination of acid and heat treatment on mAb-3 final drug substance (FDS) samples was evaluated before and after buffer exchange and concentrating. The relative quantitation of iron bound mAb species (HMW species and monomer species) detected by SEC-ICP-MS between the treatment and control groups was normalized to the relative quantitation of mAb-3 detected by SEC-UV. As shown in FIG. 25A, before buffer exchange and concentrating, acid and heat treatment correlated to an about 69% reduction in mAb-Fe species as detected by SEC-ICP-MS (top panel), and an about 1% reduction as detected by UV (bottom panel). As shown in FIG. 25B, after buffer exchange and concentrating, acid and heat treatment correlated to a reduction of about 74% in mAb-Fe species as detected by SEC-ICP-MS (top panel), and a reduction of about 28% as detected by UV (bottom panel). Thus, acid and heat treatment correlated with a reduction of about 70% in mAb-Fe species. The treatment group showed 26% as much mAb-Fe as the control group, and 78% as much total mAb, and therefore the abundance of mAb-Fe was about 33% of the control group when normalized to total mAb.

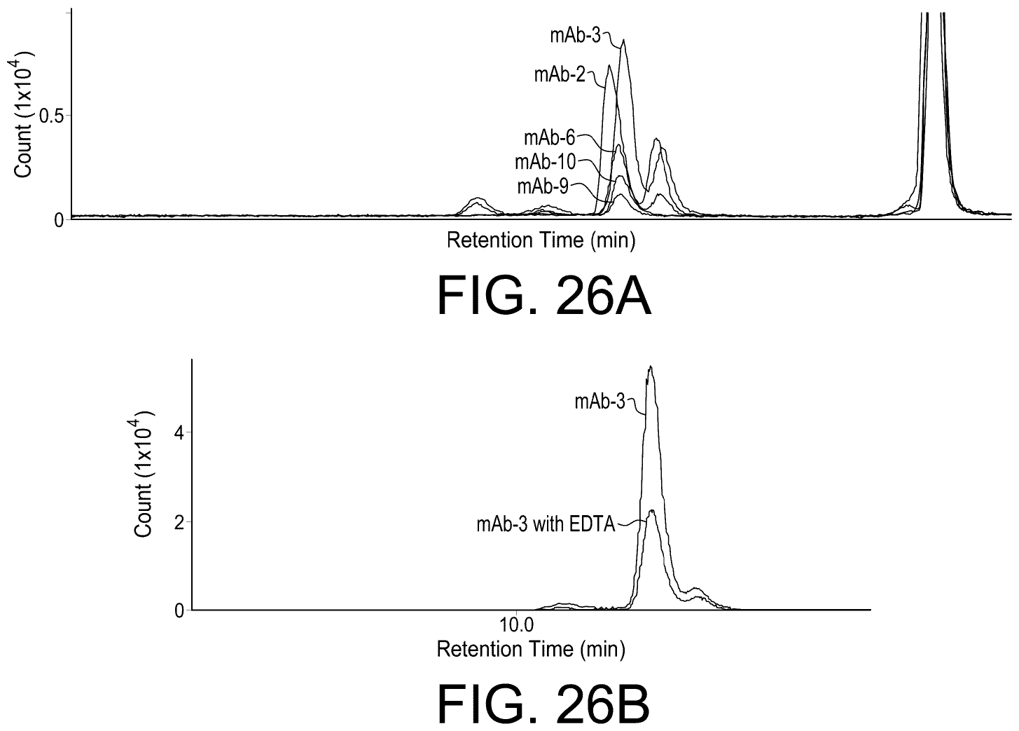

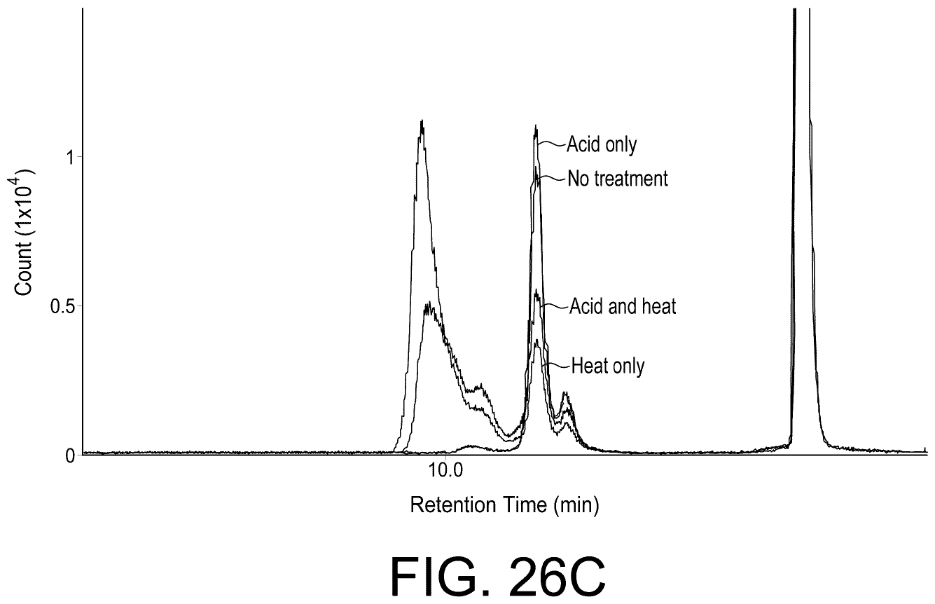

In addition to iron, copper binding and effects of EDTA treatment on copper-mAb complexes was also investigated. 200 μg amounts of IgG1 antibodies mAb-2, mAb-3, and mAb-6, and IgG4 antibodies mAb-9 and mAb-10 were injected for LC-ICP-MS-HRMS. As shown in FIG. 26A, extracted ion chromatograms for the various mAb samples showed differing amounts of monomer-Cu complexes. The effect of EDTA treatment on copper binding in mAb-3 samples were further evaluated and shown in FIG. 26B, and the effects of acid and heat treatment on copper binding in mAb-3 samples were further evaluated and shown in FIG. 26C. Unlike the mAb-Fe species, EDTA treatment did correlate with a reduction in mAb-Cu species (FIG. 26B). Acid treatment had no impact on mAb-Cu species (FIG. 26C). Unlike with mAb-Fe species, heat treatment did not cause dissociation of mAb-Cu species; rather, copper continued to be bound to denatured mAb species after heat treatment (FIG. 26C).

The investigation of treatment conditions for dissociating metal-mAb species showed that detectability using the described methods depended on the protein and the metal of interest in a sample. The LC-UV-ICP-MS-HRMS methods and systems of the present disclosure allow for careful analysis of conditions that impact metal-mAb interactions and may help prevent aggregation, truncation, oxidation, and other adverse effects of trace metals or other elements on mAb products.

### Example 7: Investigation of Sources of Trace Metal Contamination

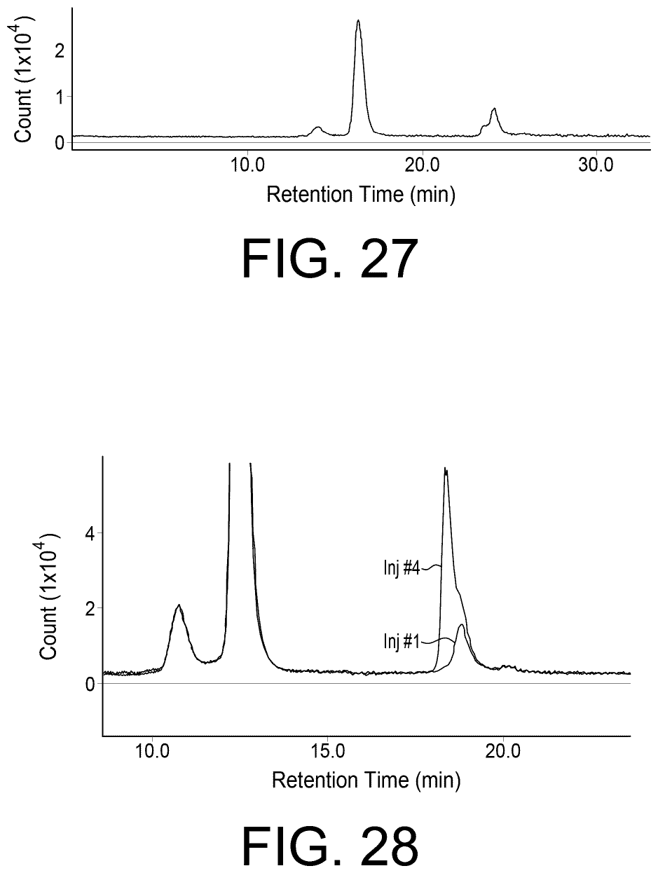

The methods and systems of the present disclosure were further applied to investigate sources of trace metal contamination in mAb samples, such as process contamination. For example, an iron chromatogram of a mAb-3 sample shown in FIG. 27 was observed to include iron-bound small molecules, including citrate, eluting at around 24 minutes. To investigate sources of the small molecule-Fe species, two samples of mAb-3 were prepared and put in an autosampler at the same time. Sample 1 was injected into an ICP-MS system four times (Inj #1, Inj #2, Inj #3, Inj #4), and Sample 2 was injected into the ICP-MS system two times (Inj #1, Inj #2).

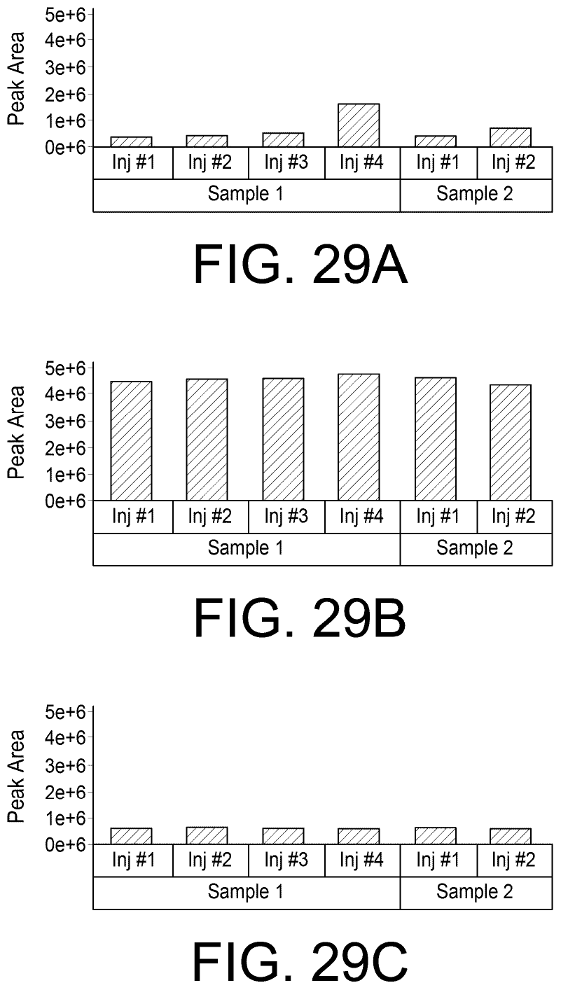

As shown in FIG. 28, which shows iron chromatograms of Inj #1 and Inj #4 of sample 1, the quantity of small molecule-Fe species eluting at around 18 minutes was greater for Inj #4 than for Inj #1. Peak areas for small molecule-Fe species across injections for both samples are shown in FIG. 29A, confirming that the amount of small-molecule-Fe species increased as the number of injections increased, across both samples. By contrast, the quantity of monomer-Fe species (FIG. 29B) and HMW-Fe species (FIG. 29C) remained relatively the same as the number of injections increased, across both samples. Changing to a new vial of sample decreased the quantity of small molecule-Fe detected (data not shown). Therefore, the variation of small molecule-Fe between samples was determined to be caused by an interaction with the stainless-steel injection needle, instead of storage time in the autosampler. At the same time, no effect of the injection needle was observed with regard to mAb-Fe species.

Thus, the methods and systems of the present disclosure were useful for sensitive analysis of trace metal contamination, even independently of mAb-Fe interactions. Given the discovery of trace metal contamination from the injection needle of the LC system, metal contamination could be reduced or eliminated through the use of a metal-free inert LC system.

### Example 8: Further Applications of LC-ICP-MS-HRMS Analyses

In addition to the examples disclosed above, the methods and systems of the present disclosure may be used with other chromatography modalities for other applications. Ion exchange chromatography (IEX)-ICP-MS-HRMS may be used to study protein-metal binding, or to identify hydrated and weakly complexed transition metal ions.

For example, SAX-ICP-MS was used to separate copper-bound albumin (“Serum Alb-Cu”) and copper-bound ceruloplasmin (“Serum Cp-Cu”) in a serum sample for a gene therapy study for Wilson's disease. As controls, protein standards for human albumin (“Control Alb-Cu”) and human ceruloplasmin (“Control Cp-Cu”) were also tested.

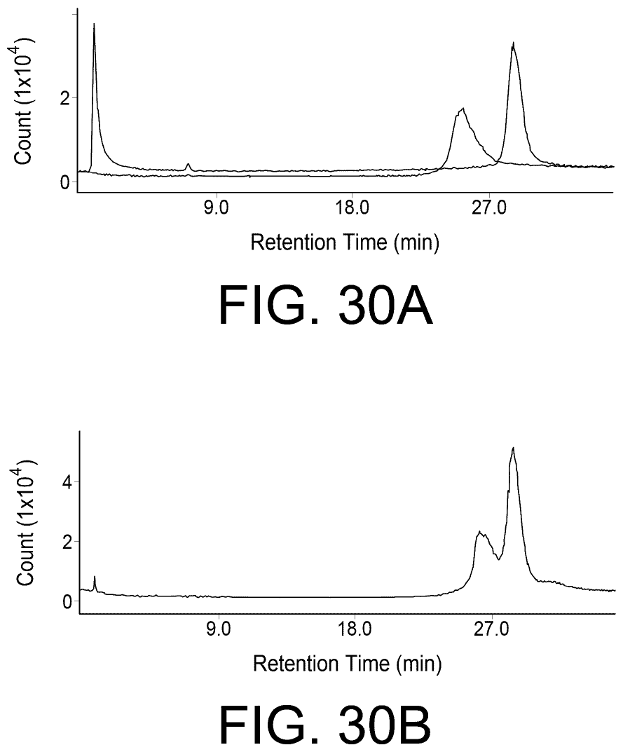

As shown in FIG. 30A, copper chromatograms for protein standards indicated a peak corresponding to Control Alb-Cu eluting at around 26 minutes, and a peak corresponding to Control Cp-Cu eluting at around 28 minutes. A peak corresponding to free copper in the ceruloplasmin standard eluted at around 1.5 minutes. As shown in FIG. 30B, copper chromatograms for the serum sample indicated the Serum Alb-Cu peak eluting at around 26 minutes and the Serum Cp-Cu peak eluting at around 28 minutes, indicating successful separation of copper-bound albumin and copper-bound ceruloplasmin in the serum sample.

ICP-MS may also be coupled with, for example, reversed phase (RP) or hydrophilic interaction chromatography (HILIC), which may be useful for studying peptide-bound metals or metalloproteomics (including, but not limited to, biological samples: cell extract, blood, tissue, sputum, etc.) to investigate protein-metal interactions in a biological system.

Embodiments of the present disclosure may further be understood by reference to the following items:

Item 1. A method for characterizing at least one metal in a sample including a polypeptide of interest, comprising:

(a) subjecting a sample including a polypeptide of interest to liquid chromatography (LC) separation to form an eluate;

(b) subjecting said eluate to inductively coupled plasma mass spectrometry (ICP-MS) analysis to identify said at least one metal;

(c) subjecting said eluate to high-resolution mass spectrometry (HRMS) analysis in parallel to the subjecting of step (b) to identify said polypeptide of interest; and

(d) comparing the results of steps (b) and (c) to characterize said at least one metal,

wherein a system performing said LC separation is coupled to a system performing said ICP-MS analysis and a system performing said HRMS analysis using a three-way splitter.

Item 2. A method for characterizing at least one metal-bound molecule in a sample including a polypeptide of interest, comprising:

(a) subjecting a sample including a polypeptide of interest to liquid chromatography (LC) separation to form an eluate, wherein said sample includes at least one metal-bound molecule;

(b) subjecting said eluate to inductively coupled plasma mass spectrometry (ICP-MS) analysis to identify at least one metal;

(c) subjecting said eluate to high-resolution mass spectrometry (HRMS) analysis in parallel to the subjecting of step (b) to identify at least one molecule having substantially the same retention time as said at least one metal; and

(d) characterizing said at least one metal-bound molecule using the results of step (b) and step (c),

wherein a system performing said LC separation is coupled to a system performing said ICP-MS analysis and a system performing said HRMS analysis using a three-way splitter.

Item 3. The method of item 1, wherein characterizing said at least one metal comprises determining or identifying said at least one metal.

Item 4. The method of item 1, wherein characterizing said at least one metal comprises determining an ionic charge of said at least one metal.

Item 5. The method of item 1, wherein characterizing said at least one metal comprises determining an isotope of said at least one metal.

Item 6. The method of item 1, wherein characterizing said at least one metal comprises quantifying said at least one metal, optionally wherein characterizing said at least one metal comprises quantifying said at least one metal for at least two retention times.

Item 7. The method of item 1, wherein characterizing said at least one metal comprises identifying and/or quantifying at least one molecule bound to said at least one metal, optionally wherein said at least one molecule comprises said polypeptide of interest, at least one HMW species of said polypeptide of interest, at least one LMW species of said polypeptide of interest, and/or at least one small molecule.

Item 8. The method of item 1, wherein characterizing said at least one metal comprises quantifying an abundance of free metal, small molecule-bound metal, and/or polypeptide-bound metal.

Item 9. The method of item 2, wherein said at least one metal-bound molecule comprises said polypeptide of interest, at least one HMW species of said polypeptide of interest, at least one LMW species of said polypeptide of interest, and/or at least one small molecule.

Item 10. The method of item 2, wherein said at least one metal-bound molecule is a polypeptide and characterizing said at least one metal-bound molecule comprises determining an amino acid sequence of said at least one metal-bound molecule.

Item 11. The method of item 2, wherein characterizing said at least one metal-bound molecule comprises quantifying said at least one metal-bound molecule.

Item 12. The method of item 2, wherein characterizing said at least one metal-bound molecule comprises determining a structure of said at least one metal-bound molecule.

Item 13. The method of item 2, wherein said at least one metal-bound molecule is a truncated protein and characterizing said at least one metal-bound molecule comprises determining a clipping site of said at least one metal-bound molecule.

Item 14. The method of any one of items 1 or 3-8, further comprising subjecting said eluate to ultraviolet detection or fluorescence detection prior to subjecting said eluate to ICP-MS analysis.

Item 15. The method of item 14, further comprising characterizing said at least one metal by comparing the results of said ultraviolet detection or fluorescence detection to the results of said ICP-MS analysis and/or said HRMS analysis.

Item 16. The method of any one of items 2 or 9-13, further comprising subjecting said eluate to ultraviolet detection or fluorescence detection prior to subjecting said eluate to ICP-MS analysis.

Item 17. The method of item 16, further comprising characterizing said at least one metal-bound molecule by comparing the results of said ultraviolet detection or fluorescence detection to the results of said ICP-MS analysis and/or said HRMS analysis.

Item 18. The method of item 1, further comprising comparing said characterization of said at least one metal in a first sample to a characterization of at least one metal in a second sample.

Item 19. The method of item 2, further comprising comparing said characterization of said at least one metal-bound molecule in a first sample to a characterization of at least one metal-bound molecule in a second sample.

Item 20. The method of item 18 or 19, wherein said first sample has not been subjected to treatment to dissociate metal-polypeptide interactions and said second sample has been subjected to treatment to dissociate metal-polypeptide interactions.

Item 21. The method of item 20, wherein said treatment comprises incubating said second sample with a chelator, subjecting said second sample to acidic conditions, and/or subjecting said second sample to heating conditions.

Item 22. The method of item 21, wherein incubating said second sample with a chelator comprises incubating said second sample with ethylenediaminetetraacetic acid, optionally wherein said incubating is performed for about one hour.

Item 23. The method of item 21, wherein subjecting said second sample to acidic conditions comprises incubating said second sample with acetic acid, optionally wherein said acetic acid is at a concentration of about 5 mM.

Item 24. The method of item 21, wherein subjecting said second sample to heating conditions comprises subjecting said second sample to temperatures from 50 to 100° C., from 70 to 90° C., about 70° C., about 75° C., about 80° C., about 85° C., or about 90° C. for from 1 to 30 minutes, from 5 to 20 minutes, about 5 minutes, about 10 minutes, about 15 minutes, or about 20 minutes.

Item 25. The method of item 18 or 19, wherein said first sample has been subjected to fewer injections in said system performing said LC separation compared to said second sample.

Item 26. The method of item 18 or 19, wherein said first sample is from a first step in a production process for said polypeptide of interest and said second sample is from a second step in said production process for said polypeptide of interest.

Item 27. The method of item 18 or 19, wherein said first sample includes a first polypeptide of interest and said second sample includes a second polypeptide of interest.

Item 28. The method of any one of items 1-27, wherein said polypeptide of interest is a therapeutic polypeptide and said sample is a sample from a production or purification process, a chromatography pool, a final concentrated pool, a drug substance, or a drug product.

Item 29. The method of any one of items 1-27, wherein said sample is a biological sample, optionally wherein said biological sample is a serum sample.

Item 30. The method of any one of items 1-29, wherein said at least one metal is a contaminant, a metal component of a cell culture medium, a metal contaminant from manufacturing equipment, a metal component of a formulation, and/or a metal contaminant from a storage or transportation container.

Item 31. The method of any one of items 1-30, wherein said at least one metal is selected from a group consisting of vanadium, iron, cobalt, nickel, copper, zinc, magnesium, aluminum, calcium, titanium, manganese, molybdenum, tungsten, sodium, potassium, cadmium, chromium, silver, or combinations thereof.

Item 32. The method of any one of items 1-31, wherein said polypeptide of interest is an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, or a variant thereof.

Item 33. The method of any one of items 1-32, wherein said liquid chromatography separation comprises reversed phase liquid chromatography, ion exchange chromatography, anion exchange chromatography, strong anion exchange chromatography, cation exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

Item 34. The method of any one of items 1-33, wherein said liquid chromatography separation is performed under native conditions.

Item 35. The method of any one of items 1-34, wherein a nebulizer for said ICP-MS analysis is a quartz concentric nebulizer.

Item 36. The method of any one of items 1-35, wherein an internal diameter (I.D.) of tubing connecting said three-way splitter to said system performing said ICP-MS analysis is from 0.1 mM to 0.2 mM, or about 0.13 mM.

Item 37. The method of any one of items 1-36, wherein said ICP-MS analysis is performed in oxygen mode or helium mode.

Item 38. The method of any one of items 1-37, wherein said system performing said HRMS analysis comprises an electrospray ionization mass spectrometer, nano-electrospray ionization mass spectrometer, or a triple quadrupole mass spectrometer.

Item 39. The method of any one of items 1-37, wherein said system performing said HRMS analysis comprises a microflow-nanospray electrospray ionization source.

Item 40. The method of any one of items 1-39, wherein said HRMS analysis is performed with a mass range of about 60-1000 m/z and/or about 1000-6000 m/z.

Item 41. The method of any one of items 1-40, wherein said sample comprises size variants of said polypeptide of interest.

Item 42. The method of any one of items 1-41, wherein said sample comprises high molecular weight (HMW) species and/or low molecular weight (LMW) species of said polypeptide of interest.

Item 43. The method of any one of items 1-42, wherein said sample comprises at least one small molecule, optionally wherein said at least one small molecule is histidine, citrate, or sucrose.

Item 44. The method of any one of items 1-43, wherein said three-way splitter directs a majority of eluate to said system performing ICP-MS analysis, optionally wherein a ratio of eluate directed to said system performing HRMS analysis to eluate directed to said system performing ICP-MS analysis is from 1:5 to 1:50, from 1:10 to 1:30, about 1:10, about 1:15, about 1:20, about 1:25, or about 1:30.

Item 45. The method of any one of items 1-44, further comprising subjecting said sample to digestion conditions prior to said LC separation.

Item 46. The method of item 45, wherein subjecting said sample to digestion conditions comprises contacting said sample to at least one digestive enzyme.

Item 47. The method of item 46, wherein said at least one digestive enzyme is selected from a group consisting of pepsin, trypsin, Tryp-N, chymotrypsin, Lys-N, Lys-C, Asp-N, Arg-C, Glu-C, papain, IdeS, or a variant thereof.

Item 48. The method of any one of items 1-47, wherein said sample comprises fragments or subunits of said polypeptide of interest.

Item 49. The method of item 48, wherein said polypeptide of interest is an antibody or antibody-derived protein and said fragments or subunits comprise Fab fragments, Fab′ fragments, Fab2 fragments, F(ab′)2 fragments, Fc fragments, Fc/2 fragments, Fv fragments, Fd fragments, and/or Fd′ fragments.

Item 50. A system for characterizing at least one metal in a sample including a polypeptide of interest, comprising a liquid chromatography system capable of receiving a sample including a polypeptide of interest coupled to a first arm of a three-way splitter, wherein a second arm of said three-way splitter is coupled to an ICP-MS system capable of identifying said at least one metal and a third arm of said three-way splitter is coupled to a HRMS system capable of identifying said polypeptide of interest.

Item 51. A system for characterizing at least one metal-bound molecule in a sample including a polypeptide of interest, comprising a liquid chromatography system capable of receiving a sample including a polypeptide of interest coupled to a first arm of a three-way splitter, wherein a second arm of said three-way splitter is coupled to an ICP-MS system capable of identifying at least one metal and a third arm of said three-way splitter is coupled to a HRMS system capable of identifying at least one molecule having substantially the same retention time as said at least one metal.

Item 52. The system of item 50, wherein characterizing said at least one metal comprises determining or identifying said at least one metal.

Item 53. The system of item 50, wherein characterizing said at least one metal comprises determining an ionic charge of said at least one metal.

Item 54. The system of item 50, wherein characterizing said at least one metal comprises determining an isotope of said at least one metal.

Item 55. The system of item 50, wherein characterizing said at least one metal comprises quantifying said at least one metal, optionally wherein characterizing said at least one metal comprises quantifying said at least one metal for at least two retention times.

Item 56. The system of item 50, wherein characterizing said at least one metal comprises identifying and/or quantifying at least one molecule bound to said at least one metal, optionally wherein said at least one molecule comprises said polypeptide of interest, at least one HMW species of said polypeptide of interest, at least one LMW species of said polypeptide of interest, and/or at least one small molecule.

Item 57. The system of item 50, wherein characterizing said at least one metal comprises quantifying an abundance of free metal, small molecule-bound metal, and/or polypeptide-bound metal.

Item 58. The system of item 51, wherein said at least one metal-bound molecule comprises said polypeptide of interest, at least one HMW species of said polypeptide of interest, at least one LMW species of said polypeptide of interest, and/or at least one small molecule.

Item 59. The system of item 51, wherein said at least one metal-bound molecule is a polypeptide and characterizing said at least one metal-bound molecule comprises determining an amino acid sequence of said at least one metal-bound molecule.

Item 60. The system of item 51, wherein characterizing said at least one metal-bound molecule comprises quantifying said at least one metal-bound molecule.

Item 61. The system of item 51, wherein characterizing said at least one metal-bound molecule comprises determining a structure of said at least one metal-bound molecule.

Item 62. The system of item 51, wherein said at least one metal-bound molecule is a truncated protein and characterizing said at least one metal-bound molecule comprises determining a clipping site of said at least one metal-bound molecule.

Item 63. The system of any one of items 50-62, further comprising an ultraviolet detector, fluorescence detector, or diode array detector coupled to said liquid chromatography system.

Item 64. The system of any one of items 50-63, wherein said at least one metal is a contaminant, a metal component of a cell culture medium, a metal contaminant from manufacturing equipment, a metal component of a formulation, and/or a metal contaminant from a storage or transportation container.

Item 65. The system of any one of items 50-64, wherein said at least one metal is selected from a group consisting of vanadium, iron, cobalt, nickel, copper, zinc, magnesium, aluminum, calcium, titanium, manganese, molybdenum, tungsten, sodium, potassium, cadmium, chromium, silver, or combinations thereof.

Item 66. The system of any one of items 50-65, wherein said polypeptide of interest is an antibody, a monoclonal antibody, a multispecific antibody, a bispecific antibody, an antibody fragment, a fusion protein, a receptor fusion protein, an antibody-derived protein, an antigen-binding protein, an IgG1 antibody, an IgG4 antibody, or a variant thereof.

Item 67. The system of any one of items 50-66, wherein said liquid chromatography system comprises reversed phase liquid chromatography, ion exchange chromatography, anion exchange chromatography, strong anion exchange chromatography, cation exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

Item 68. The system of any one of items 50-67, wherein said liquid chromatography system is configured to operate under native conditions.

Item 69. The system of any one of items 50-68, wherein a nebulizer for said ICP-MS system is a quartz concentric nebulizer.

Item 70. The system of any one of items 50-69, wherein an internal diameter (I.D.) of tubing connecting said three-way splitter to said ICP-MS system is from 0.1 mM to 0.2 mM, or about 0.13 mM.

Item 71. The system of any one of items 50-70, wherein said ICP-MS system is configured to function in oxygen mode or helium mode.

Item 72. The system of any one of items 50-71, wherein said HRMS system comprises an electrospray ionization mass spectrometer, nano-electrospray ionization mass spectrometer, or a triple quadrupole mass spectrometer.

Item 73. The system of any one of items 50-71, wherein said HRMS system comprises a microflow-nanospray electrospray ionization source.

Item 74. The system of any one of items 50-73, wherein said sample comprises size variants of said polypeptide of interest.

Item 75. The system of any one of items 50-74, wherein said sample comprises high molecular weight (HMW) species and/or low molecular weight (LMW) species of said polypeptide of interest.

Item 76. The system of any one of items 50-75, wherein said sample comprises at least one small molecule, optionally wherein said at least one small molecule is histidine, citrate, or sucrose.

Item 77. The system of any one of items 50-76, wherein said three-way splitter is configured to direct a majority of eluate to said ICP-MS system, optionally wherein a ratio of eluate directed to said HRMS system to eluate directed to said ICP-MS system is from 1:5 to 1:50, from 1:10 to 1:30, about 1:10, about 1:15, about 1:20, about 1:25, or about 1:30.

Item 78. The system of any one of items 50-77, wherein said sample comprises fragments or subunits of said polypeptide of interest.

Item 79. The system of item 78, wherein said polypeptide of interest is an antibody or antibody-derived protein and said fragments or subunits comprise Fab fragments, Fab′ fragments, Fab2 fragments, F(ab′)2 fragments, Fc fragments, Fc/2 fragments, Fv fragments, Fd fragments, and/or Fd′ fragments.

1. A method for characterizing at least one metal in a sample including a polypeptide of interest, comprising:
subjecting a sample including a polypeptide of interest to liquid chromatography (LC) separation to form an eluate, wherein the sample includes at least one metal-bound molecule;
subjecting the eluate to inductively coupled plasma mass spectrometry (ICP-MS) analysis to identify at least one metal;
subjecting the eluate to high-resolution mass spectrometry (HRMS) analysis to identify the polypeptide of interest; and
comparing results of the ICP-MS analysis and the HRMS analysis to characterize the at least one metal.

2. The method of claim 1, wherein the ICP-MS analysis and the HRMS analysis are performed in parallel.

3. The method of claim 1, wherein characterizing the at least one metal comprises determining an isotope of the at least one metal.

4. The method of claim 1, wherein characterizing the at least one metal comprises determining an ionic charge of the at least one metal.

5. The method of claim 1, wherein characterizing the at least one metal comprises quantifying an abundance of free metal, small molecule-bound metal, polypeptide-bound metal, or a combination thereof.

6. The method of claim 1, wherein characterizing the at least one metal-bound molecule comprises quantifying the at least one metal-bound molecule.

7. The method of claim 1, wherein characterizing the at least one metal-bound molecule comprises determining a structure of the at least one metal-bound molecule.

8. The method of claim 1, wherein the at least one metal-bound molecule comprises the polypeptide of interest, at least one high molecular weight (HMW) species of the polypeptide of interest, at least one low molecular weight (LMW) species of the polypeptide of interest, at least one small molecule, or a combination thereof.

9. The method of claim 1, wherein the at least one metal-bound molecule comprises a polypeptide, and characterizing the at least one metal-bound molecule comprises determining an amino acid sequence of the polypeptide.

10. The method of claim 1, wherein the at least one metal-bound molecule is a truncated protein, and characterizing the at least one metal-bound molecule comprises determining a clipping site of the at least one metal-bound molecule.

11. The method of claim 1, further comprising, prior to subjecting the eluate to ICP-MS analysis, subjecting the eluate to ultraviolet detection or fluorescence detection.

12. The method of claim 1, further comprising, prior to subjecting the same to LC separation, subjecting the sample to digestion conditions.

13. The method of claim 12, wherein subjecting the sample to digestion conditions comprises contacting the sample to at least one digestive enzyme.

14. The method of claim 1, wherein the sample comprises fragments or subunits of said polypeptide of interest.

15. The method of claim 1, wherein the sample is a biological sample.

16. The method of claim 1, wherein the at least one metal is vanadium, iron, cobalt, nickel, copper, zinc, magnesium, aluminum, calcium, titanium, manganese, molybdenum, tungsten, sodium, potassium, cadmium, chromium, silver, or a combination thereof.

17. The method of claim 1, wherein the polypeptide of interest is an antibody, a monoclonal antibody, a multispecific antibody, a bispecific antibody, an antibody fragment, a fusion protein, a receptor fusion protein, an antibody-derived protein, an antigen-binding protein, or a variant thereof.

18. The method of claim 1, wherein the polypeptide of interest is dupilumab.

19. The method of claim 1, wherein the sample includes size variants of the polypeptide of interest.

20. The method of claim 1, wherein the sample includes a high molecular weight (HMW) species of the polypeptide of interest, a low molecular weight (LMW) species of the polypeptide of interest, or a combination thereof.
